(19) United States
(12) Patent Application Publication
(10) Pub. No.: US 2006/0009805 A1
(43) Pub. Date: Jan. 12, 2006

(54) Neural stimulation device employing renewable chemical stimulation

(76) Inventors: Ralph Jensen, Norwood, MA (US); Carmen Scholz, Madison, AL (US); Luke S. Theogarajan, Somerville, MA (US)

Correspondence Address: CHOATE, HALL & STEWART LLP, TWO INTERNATIONAL PLACE, BOSTON, MA, 02110, US

(21) Appl. No.: 11/114,976

(22) Filed: Apr. 26, 2005

Related U.S. Application Data
(60) Provisional application No. 60/565,592, filed on Apr. 26, 2004.

(51) Int. Cl. A61N 1/00

(52) U.S. Cl. 607/2

(57) ABSTRACT

A variety of neural stimulation devices are disclosed. The devices comprise an uptake component comprising means for selectively transporting a stimulating species into the device; a release component comprising means for releasing the stimulating species; and means for producing a concentration gradient of a second species. The concentration gradient of the second species provides energy to transport the stimulating species into the device. The stimulating species may be an ion, e.g., a potassium ion, or a neurotransmitter. In a preferred embodiment of the invention the stimulating species is a potassium ion. In a second preferred embodiment the stimulating species is dopamine. In certain embodiments of the invention countertransport across an uptake component comprising a synthetic ABA polymer membrane is achieved using a carboxylic acid crown ether. The gradient of the second species may be provided by means of a chemical reaction that takes place inside the device. The substrate for the chemical reaction is transported into the device from the external environment. In certain embodiments the neural stimulation device comprises light-sensitive elements that comprise light-sensitive proton pumps. The proton pumps translocate protons into the device in response to light, thereby triggering release of the stimulating species. In certain embodiments the neural stimulation device comprises electronic components that receive a signal and send an activating input to the device, thereby triggering release of the stimulating species.

### CROSS-REFERENCE TO RELATED APPLICATION

This application claims priority to U.S. Provisional Patent Application 60/565,592, filed Apr. 26, 2004, the contents of which are incorporated herein by reference.

### BACKGROUND OF THE INVENTION

While many body tissues have considerable capacity for recovery after injury, neural tissue appears to possess only a limited potential for regeneration or repair. In addition, a large number of diseases affect the nervous system or neural target tissues and result in loss or absence of sensory and/or motor function. There is considerable interest in the development of devices and methods for artificial stimulation of neurons or the target cells they innervate in order to restore or provide such function or for a variety of other therapeutic purposes.

An area that has attracted considerable effort is the development of artificial vision systems, also known as visual prostheses, that could, for example, restore functional vision to the blind. These devices capture features of the visual environment and utilize the information to stimulate neurons to achieve visual sensations. Such devices offer the prospect of bypassing damaged portions of the visual system, interfacing to remaining structures in the visual pathway and producing visual sensations that would otherwise not exist. Several approaches to visual prosthesis development are currently being pursued. Retinal prostheses can be placed within the eye, either positioned on the inner surface of the retina (epi-retinal) or under the retina (sub-retinal). Alternately, visual prostheses can be placed elsewhere in the visual pathway. Retinal prostheses are of particular use in conditions characterized by damage to the retina, e.g., degeneration or loss of photoreceptor cells. Such conditions include age-related macular degeneration, which is the most common cause of blindness in individuals over age 65, and retinitis pigmentosa, which is the most common inherited cause of blindness.

In addition to visual prostheses, there is considerable interest in the development and improvement of devices to treat hearing loss such as cochlear implants. Systems to restore muscle activity after spinal cord injury, e.g., functional electrical stimulation (FES) systems are also of considerable interest. Significant efforts are also under way to develop more effective therapies for conditions such as epilepsy and Parkinson's disease by stimulating various regions within the brain, and electrical nerve stimulation is employed for the treatment of chronic pain. In addition, studies of cultured neural cells often involves applying artificial stimulation to selected neurons, and ongoing research efforts seek to develop neural networks in vitro that could be implanted into the body to restore lost neural function.

Although each of the above areas has its own specific requirements, the needs for an effective interface with neural tissue and a means to excite the neural tissue is a common feature. Current neural prosthetic devices, including visual prostheses, typically employ electrical stimulation, using electrodes to apply a voltage or current to the neuron or neuron target cell. Retinal prostheses of various types utilize microelectrode arrays produced using silicon micromanufacturing techniques or the like to apply a voltage and/or current to nerve cells in the retina. The general concept of utilizing a retinal prosthesis to restore vision to the blind was first described in U.S. Pat. No. 2,760,483 to Tassicker and later in U.S. Pat. No. 4,628,933 to Michelson, who taught the use of an epi-retinal device that utilizes both light and radiofrequency transmission. Subsequently, Humayun et al. taught the use of epi-retinal devices that use radiofrequency transmission alone. (See U.S. Pat. No. 5,935,155). Additional retinal stimulation devices, components for use in such devices, and methods for their implantation into the eye have been described, e.g., in U.S. Pat. Nos. 6,324,429; 6,120,538; 5,800,530 6,368,349; 6,075,251; 6,069,365; 6,020,593; 5,949,064; 5,895,415; 5,837,995; 5,556,423; 5,397,350; 5,597,381; 6,324,429; 5,865,839; 5,836,996; 6,389,317; and 5,944,747). Cuff electrodes are used in functional neuromuscular stimulation to electrically stimulate target tissues, and similar electrodes are being explored for stimulation of the optic nerve. Microelectrode arrays and single electrodes are used for intracortical electrical stimulation (e.g., stimulation of the visual cortex) or for stimulation of structures deeper in the brain.

Although electrical stimulation is relatively simple to implement, it has a number of disadvantages. For example, common issues that arise with electrical stimulation methods are lack of focal stimulation, biotoxicity that may result from the electrical stimulation itself, from materials present in the stimulating device or from byproducts of chemical reactions at the electrodes, and high power requirements. In addition, electrical stimulation as achieved by applying a voltage or current to neurons or neural target cells differs significantly from the mechanisms by which neural stimulation is accomplished within the body, which rely chiefly on neurotransmitters and ion fluxes across the cell membrane. Accordingly, there is a need in the art for the development of devices and alternate methods for the stimulation of neurons and neural target cells, e.g., that would not require stimulation by electrodes. In particular, there is a need in the art for development of devices and methods for the stimulation of neurons and neural target cells that would utilize ions or neurotransmitters for stimulation but would not require replenishment of the stimulating species from an external source.

### SUMMARY OF THE INVENTION

The present invention addresses these needs, among others. In one aspect, the invention provides a device for stimulation of a neuron or neuron target cell comprising an uptake component comprising means for selectively transporting a first species into the device, wherein the first species is a stimulating species; a release component comprising means for releasing the stimulating species; and means for producing a concentration gradient of a second species, wherein the concentration gradient of the second species provides energy to transport the stimulating species into the device. The stimulating species may be an ion, e.g., a potassium ion, or a neurotransmitter. In preferred embodiments of the invention the stimulating species is a potassium ion or dopamine.

The invention provides a neural stimulation device that comprises light-sensitive elements that comprise light-sensitive proton pumps. The proton pumps translocate protons into the device in response to light, thereby triggering release of the stimulating species.

The invention provides a neural stimulation device that comprises electronic components that receive a signal and send an activating input to the device, thereby triggering release of the stimulating species. In certain embodiments transport of protons in response to incident light causes release of the stimulating species.

The invention further provides a neural prosthesis comprising an array comprising a plurality of neural stimulation devices. In a preferred embodiment the neural prosthesis is a retinal prosthesis.

The invention provides a method of accumulating a stimulating species inside a neural stimulation device comprising transporting the stimulating species into the device using energy obtained from transport of a second species from inside the device to outside the device down its concentration gradient.

The invention provides a method of releasing a stimulating species from the interior of a neural stimulation device comprising: stimulating a stimulus-responsive hydrogel, thereby causing release of the stimulating species in the vicinity of a neuron or neural target cell. The stimulus may be, e.g., light. The hydrogel may be stimulated in response to an input, e.g., light, heat, or an electrical signal. Stimulation of the hydrogel causes a change in one or more properties, e.g., volume of the hydrogel. In certain embodiments the hydrogel responds to a change in pH, e.g., a change in pH caused by pumping of protons by a light-sensitive proton pump. In other embodiments the hydrogel responds to an electric field. The electric field may be applied in response to a stimulus, e.g., light, sound, motion, etc. The electric field may be applied by electronic components. The stimulating species may be contained in the hydrogel prior to release or may be in a separate compartment of the device. For example, expansion or contraction of the hydrogel may increase or decrease pressure in the separate compartment or may result in opening of an aperture.

The invention further provides methods for fabricating a neural stimulation device.

In another aspect, the invention provides a method of treating a subject in need of neural stimulation comprising implanting the neural stimulation device into the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and are not intended to be limiting. Where elements are listed in Markush group format, it is to be understood that each subgroup of these elements is also disclosed, and any element(s) can be removed from the group. Where numerical ranges are given, endpoints are included unless otherwise stated or otherwise evident from the context.

This application refers to various patents and publications. The contents of all of these are incorporated by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kandel, E., Schwartz, J. H., Jessell, T. M., (eds.), Principles of Neural Science, 4^{th} ed., McGraw Hill, 2000; and Cowan, W. M., Südhof, T. C., and Stevens, C. F., (eds.), Synapses, The Johns Hopkins University Press, Baltimore and London, 2001. In the case of conflict, the present specification will control.

### BRIEF DESCRIPTION OF THE DRAWING

The figures of the drawing, including the text therein, constitute a part of this specification and illustrate exemplary embodiments of the invention. It is to be understood that in some instances various aspects of the invention may be shown exaggerated or enlarged to facilitate an understanding of the invention.

FIG. 1A shows a schematic diagram of a frontal view of a neural stimulation device of the invention.

FIG. 1B shows a schematic diagram of a frontal view of a second neural stimulation device of the invention.

FIG. 1C shows a schematic diagram of a three-dimensional view of a neural stimulation device of the invention.

FIG. 1D shows a schematic diagram of an array of neural stimulation devices of the invention.

FIG. 1E shows a schematic diagram of a frontal view of a hybrid biochemical/electronic neural stimulation device of the invention.

FIG. 1F shows a schematic diagram of a three-dimensional view of a retinal prosthesis of the invention that incorporates an array of hybrid biochemical/electronic neural stimulation devices that interface to electronic components.

FIG. 2 shows a schematic diagram of a synthetic membrane having a structure resembling that of a lipid bilayer.

FIG. 3A shows a scheme for synthesis of a triblock ABA polymer for use in a synthetic membrane such as that in FIG. 2.

FIG. 3B shows a second scheme for synthesis of a triblock ABA polymer for use in a synthetic membrane such as that in FIG. 2.

FIG. 4 shows the mechanism of coupled countertransport of potassium ions and proteins by a crown ether carboxylic acid.

FIG. 5 shows a synthetic scheme for covalent attachment of a crown ether carboxylic acid to a PMOXA-P(DMS-co-HMS)-PMOXA membrane.

FIG. 6A shows the structure of dopamine.

FIG. 6B shows a crown ether comprising a boronic acid side chain that can be used to transport catecholamines.

FIG. 6C is a schematic diagram showing proton-coupled transport of dopamine.

FIG. 6D shows a zwitterionic dopamine boronate complex.

FIG. 6E shows structures of additional crown ether boronic acid catecholamine transporters.

FIG. 7A shows a schematic representation of passive glucose transport and the production of hydrogen ions (H⁺) inside a device of the invention.

FIG. 7B shows additional boronic acid glucose transporters.

FIG. 8A shows a synthetic scheme for covalent attachment of a glucose carrier to a PMOXA-P(DMS-co-HMS)-PMOXA membrane.

FIG. 8B shows a second synthetic scheme for covalent attachment of a glucose carrier to a PMOXA-P(DMS-co-HMS)-PMOXA membrane.

FIG. 9 shows the proton pumping mechanism of bacteriorhodopsin (from the Web site having URL anx12.bio.uci.edu/˜hudel/br/index.html)

FIG. 10 shows a process for fabrication of a neural stimulation device of the invention.

FIG. 11 shows a process for fabrication of a hybrid biochemical/electronic neural stimulation device of the invention.

FIG. 12 shows a typical record from a single cell stimulated with extracellular applicatio of K⁺. Response is shown for a cell stimulated with a 20 msec pulse of 10 mM K⁺.

FIG. 13 shows peri-stimulus histograms (PSTH) for a representative cell stimulated by increasing [K⁺] concentrations.

FIG. 14 shows dose-response curves for stimulation of retinal ganglion cells with various concentrations of K⁺.

FIG. 15 shows receptive field of a retinal ganglion cell following: a) Stimulation by elevated extracellular potassium (30 mM [K⁺]); b) Stimulation by a spot of light.

### DETAILED DESCRIPTION OF CERTAIN PREFERRED EMBODIMENTS OF THE INVENTION

I. Definitions

The following definitions are of use in understanding the invention.

“Approximately”, as used herein in reference to a number, includes numbers that fall within a range of 5% of the number in either direction (greater than or less than) unless otherwise stated or otherwise evident from the context (except where such number would exceed 100% of a possible value).

“Central nervous system” (CNS), as used herein, includes the brain, spinal cord, optic, olfactory, and auditory systems. The CNS comprises both neurons and glial cells (neuroglia), which are support cells that aid the function of neurons. Oligodendrocytes, astrocytes, and microglia are glial cells within the CNS. Oligodendrocytes myelinate axons in the CNS, while astrocytes contribute to the blood-brain barrier, which separates the CNS from blood proteins and cells. Microglial cells serve immune system functions.

“Depolarization” refers to a reduction in the absolute value of the membrane potential. Unless otherwise indicated, a “reduction in the membrane potential” refers to depolarization.

“Hyperpolarization” refers an increase in the absolute value of the membrane potential. Unless otherwise indicated, an “increase in the membrane potential” refers to hyperpolarization.

“Neural target cell or tissue” refers to a cell or tissue of any type that normally receives input from a neuron, e.g., whose activity is stimulated by or inhibited by a neuron. In particular, neural target tissues include muscle cells (e.g., skeletal muscle, cardiac muscle, or smooth muscle cells) that receive input at a neuromuscular junction and certain secretory cells, e.g., endocrine cells. Neurons themselves may be considered neural target tissues of other neurons that stimulate or inhibit them. In general, the post-synaptic cell at any synapse may be considered a neural target cell.

“Neural tissue”, as used herein, refers to one or more components of the central nervous system and peripheral nervous system. Such components include brain tissue and nerves. In general, brain tissue and nerves contain neurons (which typically comprise cell body, axon, and dendrite(s)), glial cells (e.g., astrocytes, oligodendrocytes, and microglia in the CNS; Schwann cells in the PNS). It will be appreciated that brain tissue and nerves typically also contain various noncellular supporting materials such as basal lamina (in the PNS), endoneurium, perineurium, and epineuriun in nerves, etc. Additional nonneural cells such as fibroblasts, endothelial cells, macrophages, etc., are typically also present. See [86] for further description of the structure of various neural tissues.

“Peripheral nervous system” (PNS) includes the cranial nerves arising from the brain (other than the optic and olfactory nerves), the spinal nerves arising from the spinal cord, sensory nerve cell bodies, and their processes, i.e., all nervous tissue outside of the CNS. The PNS comprises both neurons and glial cells.

“Plurality” means at least two.

A “polypeptide”, as used herein, is a chain of amino acids. A protein is a molecule composed of one or more polypeptides. A peptide is a relatively short polypeptide, typically between about 2 and 60 amino acids in length. The amino acids can be L-amino acids, D-amino acids, or unnatural amino acids (i.e., amino acids not found in nature in living organisms).

The term “small molecule”, as used herein, refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

The term “synapse” is used herein in accordance with its meaning as accepted in the art, i.e., to indicate a specialized intercellular junction between a neuron or between a neurons and another excitable cell where signals are propagated from one cell to another with high spatial precision and speed.” [De Camilli, in Cowan, supra]. Synapses are the primary sites of intercellular communication in the mammalian nervous system. Synapses may be classified as electrical or chemical, based on the mechanism by which transmission takes place. At electrical synapses, communication takes place via movement of ions through gap junctions that connect the signaling partners. At chemical synapses, communication takes place via neurotransmitter molecules that are released from a presynaptic neuron and interact with receptors on the postsynaptic cell membrane. The region between the pre and postsynaptic membranes is referred to as the synaptic cleft.

II. Overview

A neural stimulation device that employs renewable chemical stimulation is disclosed. The device utilizes either ions or neurotransmitters as stimulating species to excite a nearby neuron in response to receipt of an activating input. In preferred embodiments the device does not require storage of a large, continuously depleted internal reservoir of the stimulating species and does not require replenishment of the stimulating species from an external source. Instead, the device takes up the stimulating species or a precursor thereof from its external environment (e.g., extracellular fluid) using energy obtained either from light or from a chemical reaction that takes place within the device and utilizes a substrate that is naturally found within the body. The stimulating species is transported from the exterior to the interior of the device up its concentration gradient, i.e., from a region of lower concentration (outside the device) to a region of higher concentration (inside the device). Transport of the stimulating species is driven by an oppositely directed concentration gradient of a second species. Transport of the second species down its concentration gradient, i.e., from a region of higher concentration (inside the device) to a region of lower concentration (outside the device), provides the energy needed to transport the stimulating species into the device.

The device comprises an uptake component comprising means for selectively transporting a stimulating species into the device, a release component comprising means for releasing the stimulating species, and means for producing a concentration gradient of a second species, wherein the concentration gradient of the second species provides energy to transport the stimulating species into the device. In certain embodiments of the invention a molecular carrier transports the stimulating species into the device and transports the second species out of the device.

To facilitate understanding of the invention a simplified description of information relating to neural signal transmission is provided below. Further details are found in Kandel, supra, and Cowan, supra.

III. Neural Signaling

Neurons are the primary signaling units of the nervous system and are responsible for generating and transmitting impulses to target cells such as other neurons and various nonneuronal cells such as muscle cells. A typical neuron has a cell body, one or more dendrites, an axon, and presynaptic terminals. Dendrites are cellular processes that are the main areas for receiving signals from other neurons. The axon conveys outgoing signals to target cells. Neurons communicate among themselves and with other neural target tissues at specialized regions known as synapses. The cell that transmits the signal is known as the presynaptic cell while the cell that receives the signal is the postsynaptic cell.

Signaling in the nervous system depends in part upon the electrical properties of the cell membrane. Neurons, like other cells, maintain a difference in the electrical potential on either side of the cell membrane, which is referred to as the resting potential. In nerve cells this ranges between about −40 and −80 mV. The resting membrane potential is a consequence of (i) a difference in the concentration of various ions (including sodium ions (Na⁺), potassium ions (K⁺), chloride ions (Cl⁻), phosphate ions, amino acids, proteins, etc.), particularly Na⁺ and K⁺, on either side of the cell membrane, and (ii) a difference in the relative permeability of the cell membrane to certain of these ions, e.g., K⁺, Na⁺, and Cl⁻. The Na⁺/K⁺ pump, a membrane protein that pumps Na⁺ out of the cell and K⁺ in to the cell, keeps the intracellular Na⁺ concentration lower (e.g., about 10 times lower) than the extracellular concentration and the intracellular K⁺ concentration higher (e.g., about 20 times higher) than the extracellular concentration.

The presence of ion channels in the cell membrane that are highly permeable to K⁺ but much less permeable to Na⁺ results in selective permeability of the membrane to K⁺ while the cell is at rest. The resting membrane potential of a cell, which results from differences in the concentrations of ions inside and outside the cell and differences in the relative permeabilities of the cell membrane to these ions, is approximated by the well-known Goldman-Hodgkin-Katz equation:

$${V}_{rest} = \frac{RT}{F} ln \frac{{{P}_{K} [ {K}^{+} ]}_{out} + {{P}_{Na} [ {Na}^{+} ]}_{out} + {{P}_{Cl} [ {Cl}^{-} ]}_{in}}{{{P}_{K} [ {K}^{+} ]}_{in} + {{P}_{Na} [ {Na}^{+} ]}_{in} + {{P}_{Cl} [ {Cl}^{-} ]}_{out}}$$

This equation can be used to predict the effect of changes in the concentration of the various ions on resting membrane potential. The equation can be expanded to include additional terms representing other ions.

The signaling ability of excitable cells such as nerve and muscle cells arises from the fact that their membrane potential can be rapidly and significantly altered. The change in potential acts as a signaling mechanism. For example, a reduction in membrane potential of approximately 10 mV (e.g., from −65 mV to −55 mV) alters the membrane permeability, making the membrane much more permeable to Na⁺ than to K⁺. The change in membrane permeability that allows long-distance transmission of signals in the nervous system involves the opening of voltage-sensitive Na⁺ channels in the axon membrane, which results in an influx of Na⁺ ions and a local rise in membrane potential. The resulting flow of Na⁺ into the cell reduces the negative charge inside the cell still further, resulting in a positive feedback effect once a certain threshold of membrane potential has been reached. This depolarization, known as an action potential, is a rapid, all-or-none signal that propagates down an axon in a wave-like manner to the axon's terminals. The local rise in membrane potential spreads passively down the axon and causes adjacent regions of the membrane to reach the threshold for generating an action potential. The depolarization resulting from the opening of voltage-sensitive Na⁺ channels lasts for only a brief period of time. After a short delay the Na⁺ channels spontaneously close and voltage-sensitive K⁺ channels open, allowing exit of K⁺. This results in repolarization of the membrane and an eventual return to the original membrane potential.

When an action potential arrives at axon terminal, it initiates communication with a target cell on which the terminal ends. Cell-cell communication resulting from an action potential typically involves release of a chemical substance referred to as a neurotransmitter. Neurotransmitters are contained in Ca²⁺-sensitive vesicles near the axon terminal. Depolarization causes opening of voltage-sensitive Ca²⁺ channels, allowing entry of Ca²⁺ into the cell, which results in fusion of the vesicles with the cell membrane and consequent release of their contents into a synaptic cleft.

In addition to action potentials, which can travel for long distances, nerve cells produce local signals (e.g., receptor potentials, synaptic potentials), also resulting from changes in the membrane potential. These local signals are not actively propagated and generally decay within several millimeters. Receptor potentials are typically due to an environmental stimulus such as stretch, pressure, or light. The stimulus results, either directly or indirectly, in an alteration in ion fluxes through channels in the cell membrane. Synaptic potentials arise from the binding of neurotransmitter molecules, released from axon terminals, to receptors in the membrane of the cell on the post-synaptic side of the synapse, which results either directly or indirectly in an alteration in membrane ion fluxes. The nature of these receptors determines whether the synaptic potential will depolarizing or hyperpolarizing. Since depolarization increases the cell's ability to generate an action potential, it is generally excitatory. Conversely, since hyperpolarization reduces the cell's ability to generate an action potential it is generally inhibitory. Both excitatory and inhibitory stimulation is considered to be stimulation for purposes of the present invention.

An action potential is triggered when the membrane of an excitable cell is sufficiently depolarized as a result of inputs (e.g., receptor potentials or synaptic potentials) that propagate locally in the cell. In certain neurons and neural target cells a single synaptic potential causes sufficient depolarization to trigger an action potential. In other neurons, e.g., many in the CNS, an action potential is triggered when depolarization resulting from the summation of excitatory post-synaptic potentials and inhibitory post-synaptic potentials reaches a certain threshold.

Many different neurotransmitters and neurotransmitter receptors exist in the nervous system, and the effects of neurotransmitter binding vary depending upon the nature of the receptor. Most neurotransmitters can be classified as either small molecules (e.g., ACh); biogenic amines such as dopamine, serotonin, epinephrine, norepinephrine, etc.; amino acids such as gamma-amino butyric acid (GABA), glycine, and glutamate, or neuroactive peptides. Certain neurotransmitters can activate multiple different receptor types. Neurotransmitter receptors may themselves act as ion channels that open in response to transmitter binding or may be coupled to second messenger systems in the cell. For example, in the case of neural transmission at the neuromuscular junction in skeletal muscle, ACh released from the axon terminal of an activating neuron binds to ACh-sensitive receptors in the post-synaptic membrane. These receptors are ion channels, and binding of ACh results in opening of the channel, which allows movement of Na⁺ ions into and K⁺ ions out of the cell. The net result is membrane depolarization. Glutamate is an excitatory neurotransmitter in the CNS that binds to several types of receptors including some that conduct both Na⁺ and K⁺ ions in a similar manner to the ACh receptor while others act in an excitatory or inhibitory manner either as ion channels or by activating second messenger systems. Various inhibitory neurotransmitters (e.g., gamma-aminobutyric acid and glycine in the CNS), activate receptors that act as Cl⁻ and/or K⁺ channels. Receptors that are coupled to second messenger systems do not function as ion channels themselves. Instead, they act indirectly by altering intracellular metabolism, typically resulting in the production or activation of molecules in the cell, which then cause opening or closing of ion channels.

IV. Device for Stimulation of Neurons or Neural Target Cells

As will be appreciated from the description above, the mechanisms by which neurons and neural target cells are stimulated in the body differ greatly from those employed by current neural prosthetic devices, which rely largely on the direct application of voltage and/or current to cells, typically by means of metal electrodes that are provided with an external power source. A number of existing devices employ neurotransmitters for artificial neural stimulation in an attempt to more closely approximate natural chemical signaling mechanisms. For example, US Patent Pub. No. 20030032946 discloses an artificial synapse chip that includes a reservoir for containing a neuromodulatory agent. However, use of an internal reserve poses a major risk since neurotransmitter can be highly toxic. Furthermore, the contents of the reserve would continuously diminish and would eventually require supplementation, particularly if large amounts of the stimulating species are required. Replenishing the neurotransmitter from an external source does not avoid the risk of leakage and is also problematic, particularly in the case of devices such as retinal prostheses that may be permanently implanted. The inventors are unaware of any system that utilizes chemical stimulation and replenishes the stimulating species using energy obtained from light or a chemical reaction that utilizes a substrate naturally found in the body.

The present invention provides devices and methods for stimulating neurons and/or neural target cells by use of either ions or neurotransmitter molecules. Such devices are referred to herein as “neural stimulation devices”. Chemical stimulation is achieved through focal application of either an ion or a neurotransmitter in a manner similar to that in which release of neurotransmitter by a presynaptic cell in the body results in stimulation of the postsynaptic cell. Application of a sufficient quantity of the ion or neurotransmitter in the vicinity of an excitable cell results in firing of an action potential. Application of lesser quantities results in a local potential.

Rather than employing an initial internal supply of the stimulating species that is depleted over time and must be replenished from an external supply if exhausted, the devices of the invention replace the stimulating species by recovering it from the extracellular environment. In the case of prosthetic neural stimulation devices that are implanted into the body, the stimulating species is recovered from the extracellular fluid. In the case of neural stimulation devices that are used in settings in which neurons and/or neural target cells are cultured in vitro, e.g., as tissue slices, isolated cells, etc., the stimulating species is recovered from the tissue culture medium. The devices and methods of the invention thus employ renewable chemical stimulation, in which the stimulating species is released from the device, and ions or neurotransmitter molecules are recovered from the extracellular environment or generated within the device.

In general, such chemical stimulation, and devices and methods that employ such stimulation, is considered “renewable” if, following return to steady state (equilibrium) after release of a predetermined amount of a stimulating species (e.g., an amount sufficient to stimulate a target cell, or an amount released in response to a particular release stimulus or trigger) the amount of stimulating species recovered or generated is at least 25% of the amount released. In preferred embodiments of the invention, the amount of stimulating species recovered or generated is at least 50% of the amount released. In more preferred embodiments of the invention the amount recovered or generated is at least 75% of the amount released. In yet more preferred embodiments of the invention the amount recovered or generated is at least 90%, at least 95%, at least 98%, at least 99%, or 100% of the amount released. A device of the invention need not be fully renewable but may also include a nonrenewable source of the stimulating species. The renewable aspect of such combined devices serves to extend the useful lifespan of the device.

In general, renewing the supply of stimulating species from the extracellular environment requires sequestering the species into the device from a background solution (e.g., extracellular fluid) that contains a lower concentration of the species. The present invention encompasses the recognition that such transport should be both selective and should be able to operate in an uphill direction with respect to the concentration of the stimulating species. In other words, the device requires a means to transport the stimulating species up its concentration gradient, i.e., from a region of lower concentration (outside the device) to a region of higher concentration (inside the device). As described below, selective transport can be achieved using any of a variety of specific molecular carriers that reversibly form a complex with the neurotransmitter or ion of interest and translocate it into the device. Selection of the appropriate carrier molecule(s) depend on the particular stimulating species.

Transport of a species against its concentration gradient is thermodynamically unfavorable. To achieve uphill transport, it is therefore necessary to supply energy to the system in order to make it thermodynamically feasible. The devices of the invention supply the needed energy to transport the stimulating species into the device by maintaining an oppositely directed concentration gradient in a second species. The concentration of the second species is higher inside the device than in the fluid outside the device. Movement of the second species down its concentration gradient, i.e., from the interior of the device to the exterior, which contains a lower concentration of the second species, provides the energy needed to transport the stimulating species up its concentration gradient. Either light or a chemical reaction inside the device is used to transport the second species into the device or generate it internally. Thus the device does not rely on electrical energy from an external source to recover the stimulating species.

The invention provides a device for stimulating a neuron or neural target cell comprising an uptake component comprising means for selectively transporting a stimulating species into the device; a release component comprising means for releasing the stimulating species; and means for producing a concentration gradient of a second species, wherein the concentration gradient of the second species provides energy to transport the stimulating species into the device. In certain embodiments of the invention the release component also serves to store the stimulating species until it is released. In other embodiments a discrete storage component is provided. The uptake component includes a carrier that translocates the stimulating species across the uptake component up its concentration gradient and into the release component or into a physically distinct storage component. In certain embodiments of the invention the carrier also transports the second species out of the device down its concentration gradient. The uptake component provides structural support for the carrier.

When an appropriate activating input is received by the device, the stimulating species is released in the vicinity of a cell to be stimulated. Thus the release component is generally positioned in the vicinity of a neuron or neural target cell to be stimulated. Various embodiments of the invention employ one or more proteins that are naturally found in cells, or modified versions thereof. Such proteins may be components of the carrier system that transports the stimulating species, the energy generating system, and/or the input-receiving system. The following sections describe the uptake component, the stimulating species and corresponding carrier(s), the storage and release component(s), the energy providing means, and the input receiving means in further detail. It will be appreciated that references to a “carrier”, “molecular carrier”, “stimulating species”, “second species”, etc., can refer to either individual entities (e.g., a single molecule of a stimulating species such as a single K⁺ ion) or can refer to the species collectively, i.e., to multiple individual entities).

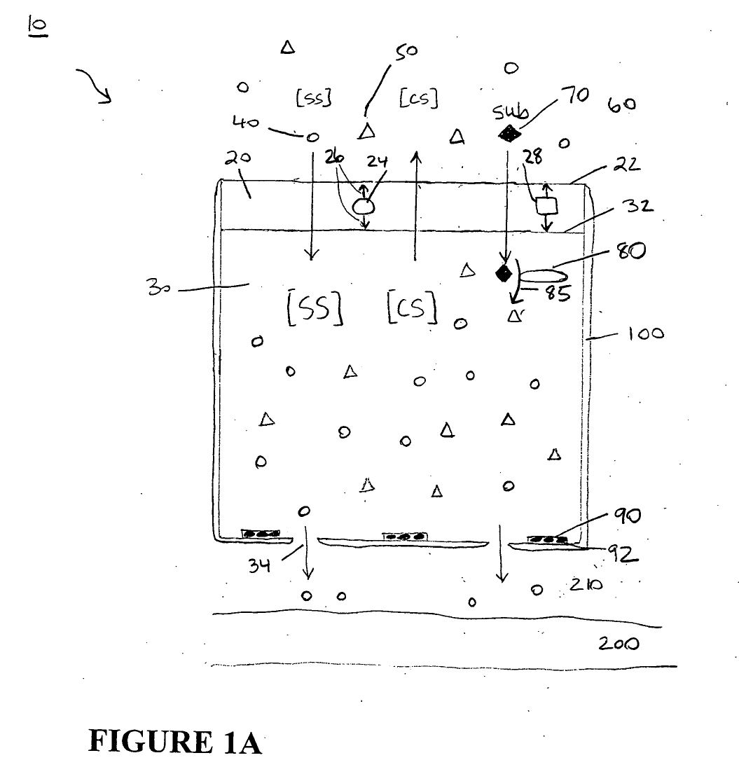

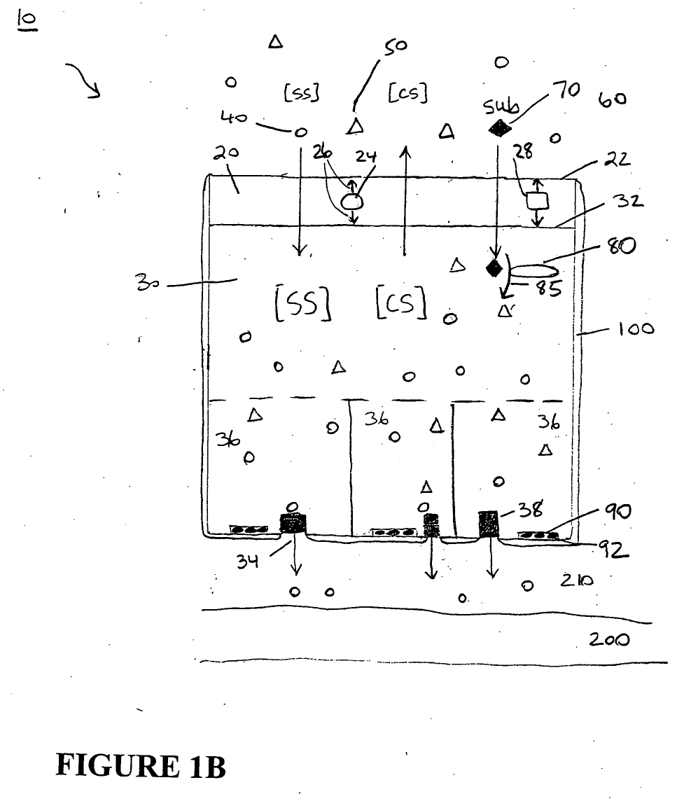

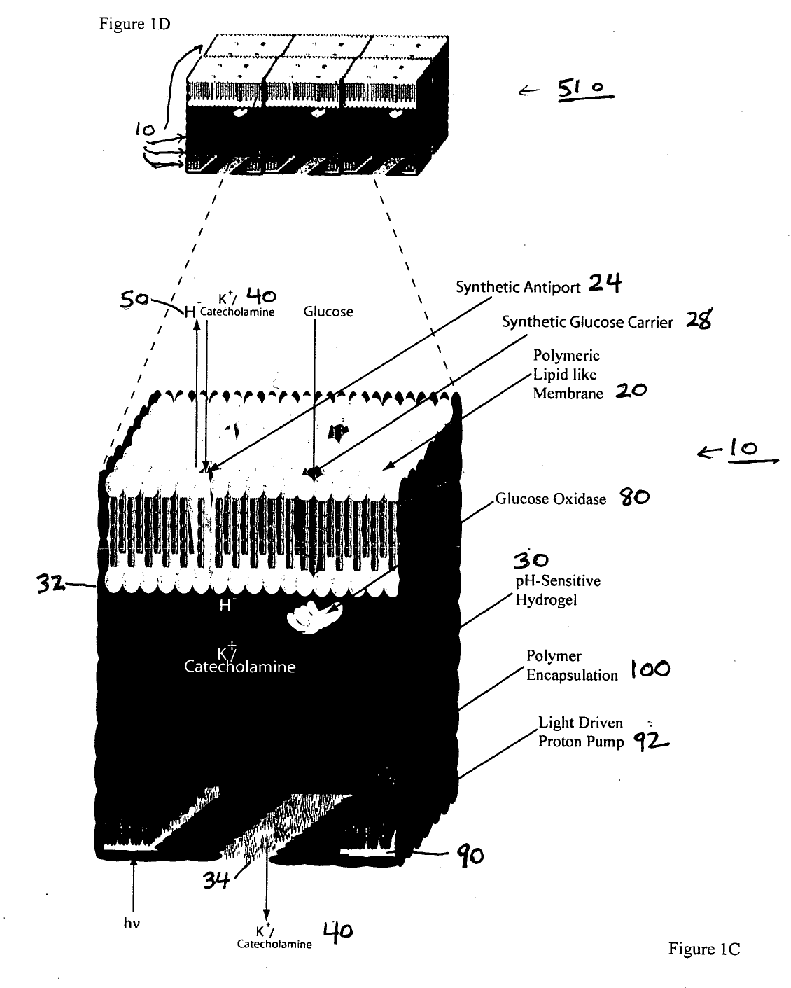

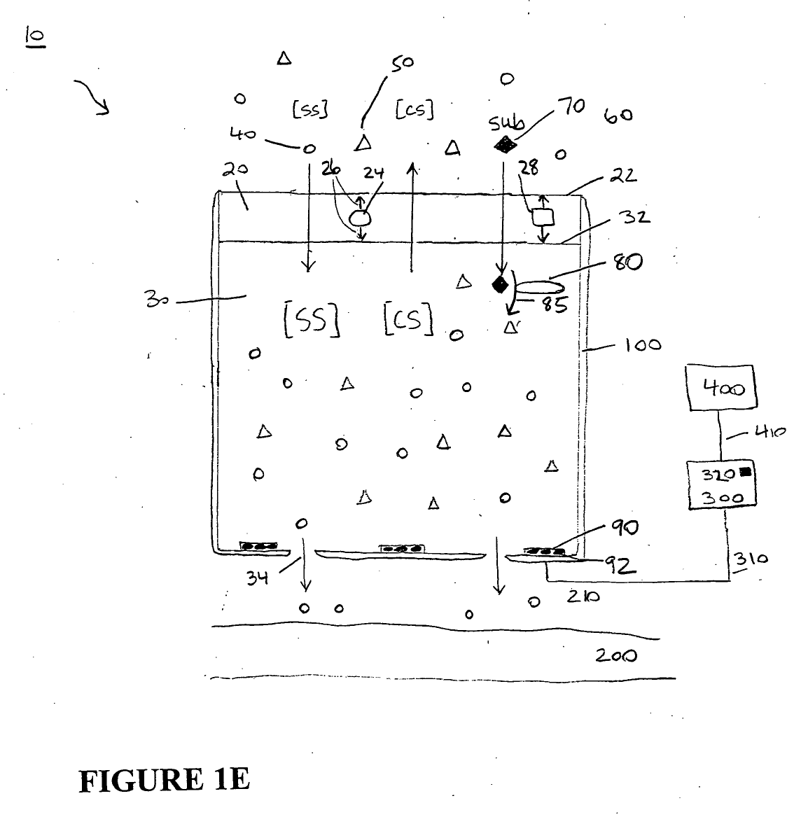

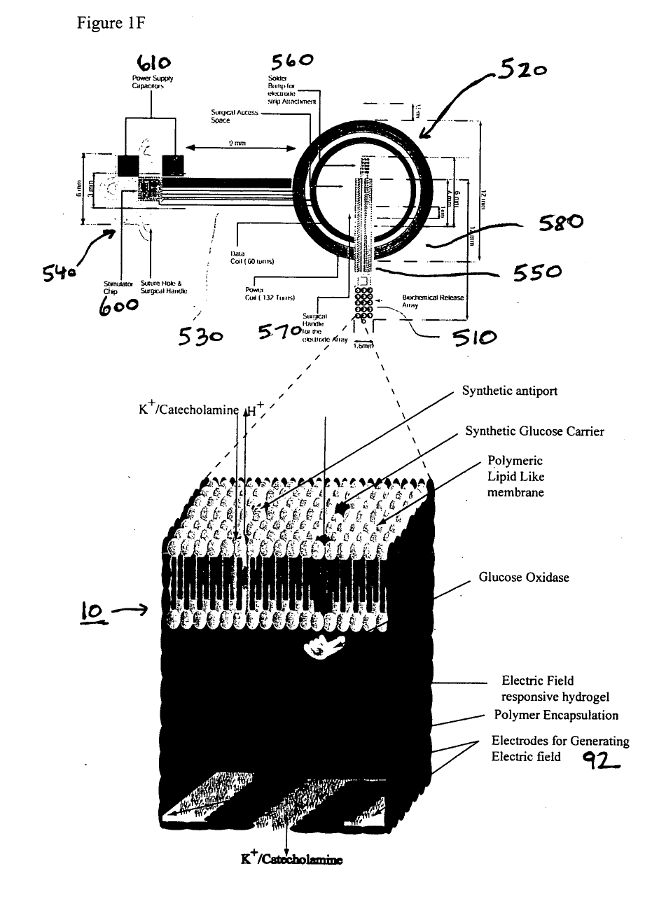

FIG. 1A is a schematic diagram showing a frontal view of a device 10 of the invention and its mechanism of action. The device comprises an uptake component 20 and a storage/release component 30. The uptake component has an interface 22 with the external environment of the device. The uptake component and the storage/release component contact one another at an interface 32. The uptake component comprises carriers 24. Carriers 24 transport a stimulating species 40 across uptake component 20 into storage/release component 30. As discussed further below, storage/release component 30 preferably comprises a stimulus-responsive hydrogel.

Stimulating species 40 has a higher concentration [SS] inside the storage/uptake component than in the external environment 60. Carriers 24 also transport a second species 50 out of the storage/release component across the uptake component to the external environment of the device. The second species is countertransported across and through the uptake component and may thus be referred to as a countertransported species. The second species has a higher concentration [CS] inside the storage/release component than in the external environment. In preferred embodiments of the invention the same carrier species transports the stimulating species and the countertransported species in opposite directions across and through the uptake component as indicated by arrows 26. In other embodiments of the invention different carrier species are used.

The uptake component also comprises carriers 28. Carriers 28 transport a substrate 70 for a chemical reaction from the external environment to the storage/release component. The storage release component comprises an enzyme 80 that catalyzes a reaction 85 to produce molecules of countertransported species 50. Production of countertransported species 50 inside the device provides the energy needed for accumulation of stimulating species 40 from the external environment. In alternate embodiments countertransported species 50 is transported into the device rather than generated within it.

In operation, e.g., when implanted into the body, at least a portion of storage/release component 30 is located in the vicinity of and preferably adjacent to a neuron or neural target cell 200 to be stimulated. Preferably storage/release component is located in the vicinity of and preferably adjacent to a cell body or dendrite. Storage/release component 30 is in communication with the external environment via apertures 34 through which stimulating species 40 is released in response to receipt of an activating input. Stimulating species 40 diffuses across space 210, which is preferably less than approximately 10-20 microns in width, to stimulate the neuron or neural target cell.

Located within or adjacent to storage/release component 30 are release triggers 90. The release triggers comprise means for sensing or receiving an activating input and means for triggering release of the stimulating species. The release trigger may comprise or consist of a light-sensitive element 92. In certain embodiments of the invention the light-sensitive element is a proton pump that absorbs a photon and pumps a proton into storage/release component 30. The increase in pH caused by an influx of protons causes release of the stimulating species. In other embodiments of the invention the release trigger comprises an electrode that receives an electrical signal. An electric field causes release of the stimulating species. The electrical signal may be generated by a variety of means as discussed further below.

In preferred embodiments of the invention the device is partially encapsulated in a protective layer 100 which may be, for example, a polymer layer. The protective layer can extend over part of interface 22.

A. Stimulating Species

As discussed above, focal application of either an ion or a neurotransmitter can artificially stimulate a neuron or neural target cell. In general, any neurotransmitter can be used in the invention, provided that the cell to be stimulated contains receptors for that neurotransmitter in its cell membrane. Any of a number of different ions may be selected as the stimulating species. Since the cellular plasma membrane is generally relatively impermeable to ions in the absence of ion channels that allow entry and/or exit of the ion, appropriate ions are those for which such channels exist in the cells to be stimulated, e.g., K⁺, Na⁺, Ca²⁺, Cl⁻.

The inventors have recognized that K⁺ is a preferred stimulating species, particularly for prosthetic devices that are to be implanted into the body for a variety of reasons. Normal homeostatic mechanisms maintain a relatively stable concentration of K⁺ in the extracellular fluid, and excitable cells such as neurons contain K⁺ channels in their cell membranes. At rest, the cell is more permeable to K⁺ than it is to Na⁺. Various neurophysiological studies of isolated cells have demonstrated that by raising the K⁺ concentration locally around a cell it is possible to depolarize the cell [2, 3, 4]. The inventors have shown that rabbit retinal tissue can be excited by focal application of K⁺[1]. The inventors have performed experiments to determine the concentration of K⁺ that is needed to excite the rabbit retina. The results indicate that a modest increase in background K⁺ concentration (˜10 mM) is sufficient to excite the firing of an action potential by retinal ganglion cells (Example 1), thereby providing suitable parameters for a neural excitation device based on K⁺ release. Furthermore, the receptive fields for both light and K⁺ evoked responses were qualitatively similar in terms of their spatial and temporal features, indicating the feasibility of using K⁺ as a stimulating species. K⁺ is used herein as an exemplary stimulating ion to illustrate the invention. However, it is to be understood that the invention encompasses the use of other ions.

Biogenic amines such as catecholamines (e.g., dopamine, epinephrine, and norepinephrine) are among the many neurotransmitters that can be employed for renewable chemical stimulation. Others include amino acids such as GABA, glycine, or glutamate, peptides, etc. Dopamine is used herein as an exemplary stimulating neurotransmitter species to illustrate the invention. However, it is to be understood that the invention encompasses the use of other neurotransmitters.

In certain embodiments of the invention multiple different stimulating species are used in the same device. For example, both an ionic species and a neurotransmitter species can be employed to stimulate a cell of interest, or two different ionic species or a combination of multiple neurotransmitters can be used.

B. Uptake Component

In certain preferred embodiments of the invention the device comprises distinct uptake and storage/release components. These components are, in general, in physical communication so that a stimulating species can be transferred from the uptake component into the storage component, which may also function as a release component. For purposes of description it is assumed that a single component, referred to hereafter as a “storage/release” component, functions as both storage component and release component. However, it is to be understood that other configurations are encompassed as described below.

At least a portion of the uptake component is in contact with a fluid in the external environment of the device. For example, when the device is implanted into the body at least a portion of the uptake component is in contact with the extracellular fluid. At least a portion of the uptake component is in contact with the storage/release component (or at least with the storage component in devices that have separate storage and release components). Thus there is at least one interface between the uptake component and the external environment and at least one interface between the uptake component and the storage/release component (or at least between the uptake component and the storage component in devices that have separate storage and release components). In a preferred embodiment the uptake and storage/release components are arranged to form a structure comprising layers that are adjacent to one another, so that one or more surfaces of the components are in contact, and at least one surface of the uptake component is in contact with the external environment.

As discussed above FIG. 1 shows a schematic diagram of an exemplary embodiment of the invention in which uptake component 20 is adjacent to storage and release component 30, so that adjoining surfaces of the two components form an interface 32. The uptake component also has an interface 22 with the external environment. In other embodiments the uptake component is at least partly embedded within the storage/release component, or the storage/release component is at least partly embedded within the uptake portion. One of the components may be entirely embedded within the other, provided that the embedded component is communication with the environment external to the device (typically the extracellular fluid or tissue culture medium), e.g., via channels.

In preferred embodiments of the invention the uptake component comprises one or more material layers and a plurality of molecular carriers. The molecular carriers transport the stimulating species into the storage/release component. The molecular carriers are discrete entities that are operably associated with a material that forms the bulk of the uptake component. By “operably associated” is meant that the molecular carriers are either noncovalently or covalently attached to a material layer that makes up at least part of the uptake component and are oriented in such a way that they function to transport a stimulating species into the device. The material layer(s) of the uptake component provide mechanical support for the carriers and also serve as a barrier to the entry of undesired species into the device. The remaining portions of the device may be largely encapsulated to prevent such entry. The molecular carriers are either synthetic or naturally derived small molecules, peptides, polypeptides, or proteins. The molecular carriers can comprise one or more molecular species, which may be the same or differerent. For example, some proteins are comprised of multiple polypeptides. Thus the carrier can be a molecule or a complex comprised of multiple molecules. In preferred embodiments a molecular carrier reversibly associates with the stimulating species and mediates its transfer across the material layer(s) of the uptake component and into the storage/release component.

Preferably the material layer(s) of the uptake component possess mechanical and chemical stability and allow for incorporation and, optionally, covalent attachment of the carriers and other molecular elements of the device. The uptake component should provide an environment that is compatible with the operation of the carriers, e.g., it should be chemically compatible with them so that they will remain stable when incorporated therein.

The molecular carriers may operate in a variety of different ways to transport the stimulating species into the storage/release component. In certain embodiments of the invention the carriers span the uptake component so that a first region of the carrier is in contact with the external environment and a second region of the carrier is in contact with the storage/release portion. In certain embodiments of the invention a carrier molecule moves back and forth within the material layer(s) of the uptake component. A carrier may associate with a molecule of the stimulating species while near the side of the uptake component that contacts the external environment and then migrate (e.g., by diffusion) across the uptake component and discharge the stimulating species into the storage/release component.

In certain embodiments of the invention a molecule of the stimulating species becomes associated with a carrier molecule at a surface of the uptake portion that is in contact with the external environment and is transferred to a second carrier molecule located more deeply within the uptake component. The second carrier molecule may have the same chemical structure as the first or may have a different structure. The stimulating species may be transferred between multiple carrier molecules until it eventually reaches a carrier molecule that is in contact with the storage/release component into which it is then discharged. In yet other embodiments the carrier has a channel-like structure, comprising a pore through which the stimulating species can pass.

In preferred embodiments of the invention the uptake component has a structure comprising two substantially planar hydrophobic layers separated by a hydrophilic layer. This structure resembles that of the plasma membrane of a cell, referred to as a lipid bilayer. For purposes of description a structure having a sheetlike configuration with a third dimension that is generally considerably smaller than the other two dimensions is referred to as a membrane.

Naturally occurring plasma membranes consist primarily of phospholipids with their nonpolar lipid tails in the interior and their polar heads interfacing with the polar water molecules outside the cell. These and other similar amphiphilic molecules self-assemble to form membrane structures. Many membranes having similar structures have been manufactured in vitro using techniques such as the “black membrane” process, the Langmuir-Blodgett process, etc. Generally, the molecular constituents from which the membrane is to be formed are dissolved in a solvent, such as chloroform and/or toluene at a low concentration (e.g., approximately 1 to 10% by weight). The amphiphilic molecules organize into their most stable structure, which is a layered membrane, with the hydrophilic portions towards the exterior (e.g., in contact with an aqueous phase) and the hydrophobic portions towards the center. See, e.g., references 51 and 52 for extensive reviews of natural and synthetic lipid bilayers.

For a variety of reasons, relatively thin membranes are preferred. For example, in embodiments in which a carrier molecule spans the uptake portion or flips from one side of the uptake portion to the other, the size of the carrier molecule establishes an upper bound for the thickness of the uptake portion. In general, if the carrier molecule diffuses across the uptake component or if the stimulating species migrates from one carrier molecule to another to transit the uptake portion, the time required for uptake will be inversely related to the square of the distance traveled. If the stimulating species migrates between multiple carrier molecules in order to be transferred, the efficiency of transfer will be reduced as the number of carrier molecules required to span the uptake component increases. Preferably the width of the membrane is between approximately 1 nm and 1 μm, more preferably between approximately 1 nm and 100 nm, and yet more preferably between approximately 5 nm and 50 nm, between approximately 5 nm and 20 nm, e.g., approximately 10 nm.

Any of a wide variety of materials can be used to form the membrane portion of the uptake component. The hydrophobic domains may be made of the same material or of different materials. In a preferred embodiment the membrane is formed from a polymeric material, optionally cross-linked to provide increased mechanical stability. Numerous synthetic polymers are known in the art. Suitable polymers include ABA copolymers in which A is a hydrophilic segment and B is a hydrophobic segment, or ABC polymers, in which A and C are hydrophilic segments and B is a hydrophobic segment. The polymers are generally block copolymers, which is understood to include linear block copolymers and other structures such as graft and comb structures. Preferably the hydrophobic layer has a relatively low glass transition temperature that allows the carriers to move freely within it and avoids the need for plasticizers. However, in certain embodiments plasticizers are included.

U.S. Pat. Nos. 5,807,944 and 6,723,814 and WO 97/49387 disclose numerous examples of suitable hydrophobic and hydrophilic polymers. Methods for making membranes from these polymers and appropriate crosslinking agents and crosslinking techniques are also described. U.S. Pat. No. 6,723,814 also discloses that biological transport proteins can be reconstituted in these membranes and function to transport species such as ions and sugars across the membranes in which they are embedded.

Exemplary hydrophobic polymers include polysiloxane such as polydimethylsiloxane and polydiphenylsiloxane, perfluoropolyether, polystyrene, polyoxypropylene, polyvinylacetate, polyoxybutylene, polyisoprene, polybutadiene, polyvinylchloride, polyalkylacrylate (PAA), polyalkylmethacrylate, polyacrylonitrile, polypropylene, PTHF, polymethacrylates, polyacrylates, polysulfones, polyvinylethers, and poly(propylene oxide), and copolymers thereof. The hydrophobic segment preferably contains a predominant amount of hydrophobic monomers. A hydrophobic monomer is a monomer that typically gives a homopolymer that is insoluble in water and can absorb less than 10% by weight of water. Suitable hydrophobic monomers are C1-C18 alkyl and C3-C18 cycloalkyl acrylates and methacrylates, C3-C18 alkylacrylamides and -methacrylamides, acrylonitrile, methacrylonitrile, vinyl C1-C18 alkanoates, C2-C18 alkenes, C2-C18 haloalkenes, styrene, (lower alkyl)styrene, C4-C12 alkyl vinyl ethers, C2-C10 perfluoro-alkyl acrylates and methacrylates and correspondingly partially fluorinated acrylates and methacrylates, C3 through C12 perfluoroalkylethylthiocarbonylaminoethyl acrylates and methacrylates, acryloxy- and methacryloxyalkylsiloxanes, N-vinylcarbazole, C1 through C12 alkyl esters of maleic acid, fumaric acid, itaconic acid, mesaconic acid, vinyl acetate, vinyl propionate, vinyl butyrate, vinyl valerate, chloroprene, vinyl chloride, vinylidene chloride, vinyltoluene, vinyl ethyl ether, perfluorohexyl ethylthiocarbonylaminoethyl methacrylate, isobornyl methacrylate, trifluoroethyl methacrylate, hexa-fluoroisopropyl methacrylate, hexafluorobutyl methacrylate, tristrimethylsilyloxysilylpropyl methacrylate (TRIS), and 3-methacryloxypropylpentamethyldisiloxane.

Exemplary hydrophobic polymers include polyoxazoline, polyethylene glycol, polyethylene oxide, polyvinyl alcohol, polyvinylpyrrolidone, polyacrylamide, poly(meth)acrylic acid, polyethylene oxide-co-polypropyleneoxide block copolymers, poly (vinylether), poly(N,N-dimethylacrylamide), polyacrylic acid, polyacyl alkylene imine, polyhydroxyalkylacrylates such as hydroxyethyl methacrylate (HEMA), hydroxyethyl acrylate, and hydroxypropyl acrylate, polyols, and copolymeric mixtures of two or more of the above mentioned polymers, natural polymers such as polysaccharides and polypeptides, and copolymers thereof, and polyionic molecules such as polyallylammonium, polyethyleneimine, polyvinylbenzyltrimethylammonium, polyaniline, sulfonated polyaniline, polypyrrole, and polypyridinium, polythiophene-acetic acids, polystyrenesulfonic acids, zwitterionic molecules, and salts and copolymers thereof. The hydrophilic segment preferably contains a predominant amount of hydrophilic monomers. A hydrophilic comonomer is a monomer that typically gives a homopolymer that is soluble in water or can absorb at least 10% by weight of water. Suitable hydrophilic monomers are hydroxyl-substituted lower alkyl acrylates and methacrylates, acrylamide, methacrylamide, (lower alkyl) acrylamides and methacrylamides, N,N-dialkyl-acrylamides, ethoxylated acrylates and methacrylates, polyethyleneglycol-mono methacrylates and polyethyleneglycolmonomethylether methacrylates, hydroxyl-substituted (lower alkyl) acrylamides and methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, vinylically unsaturated carboxylic acids having a total of 3 to 5 carbon atoms, amino(lower alkyl)-(where the term amino also includes quaternary ammonium), mono(lower alkylamino)(lower alkyl) and di(lower alkylamino)(lower alkyl) acrylates and methacrylates, allyl alcohol, 3-trimethylammonium 2-hydroxypropylmethacrylate chloride (Blemer, QA, for example from Nippon Oil), dimethylaminoethyl methacrylate (DMAEMA), dimethylaminoethylmethacrylamide, glycerol methacrylate, and N-(1,1-dimethyl-3-oxobutyl)acrylamide.

The segments A and B, or A, B, and C, are linked together through a bond that may be hydrolyzable or non-hydrolyzable. A non-hydrolyzable bond is a covalent bond that is not cleaved by an ordinary aqueous or solvent hydrolysis reaction, e.g. under acidic or basic conditions. Specific bonds that are hydrolyzable within the meaning of the term are well known to those skilled in the art. A non-hydrolyzable bond between segments A and B in the amphiphilic segmented copolymer can be formed by polymerizing a suitable hydrophilic monomer (from segment A) in the presence of a suitably functionalized hydrophobic monomer (from segment B) such that a block of units of the hydrophilic monomer grows from the site of functionalization of the hydrophilic monomer or, alternatively by polymerizing a suitable hydrophobic monomer in the presence of a suitably functionalized hydrophilic monomer such that a block of units of the hydrophobic monomer grows from the site of functionalization of the hydrophilic monomer.

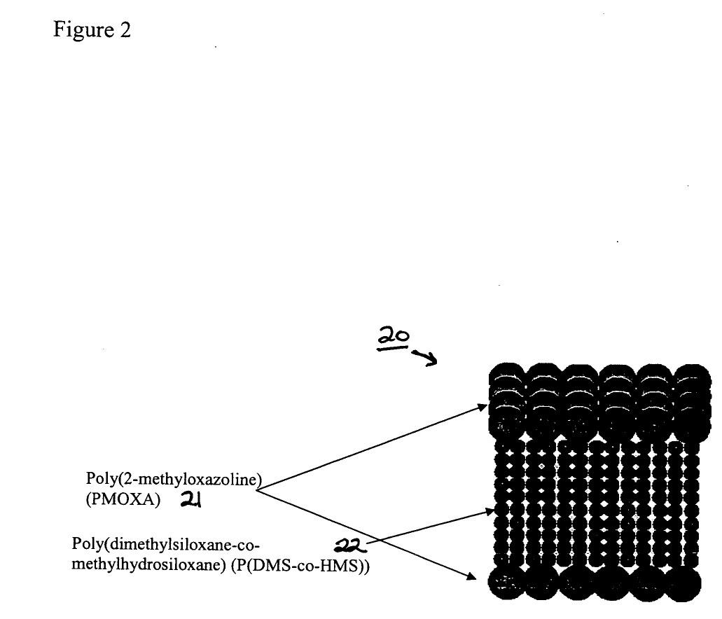

In a preferred embodiment a PMOXA-P(DMS-co-HMS)-PMOXA membrane, also referred to as a PMOXA-PDMS-PMOXA membrane is used (U.S. Pat. No. 6,723,814 and references 6-8. FIG. 2 shows a schematic of the layer structure of an uptake component 20 comprising a membrane. As shown therein, uptake component 20 comprises hydrophilic layers 21 (the A segment) and hydrophobic layer 23 (the B segment).

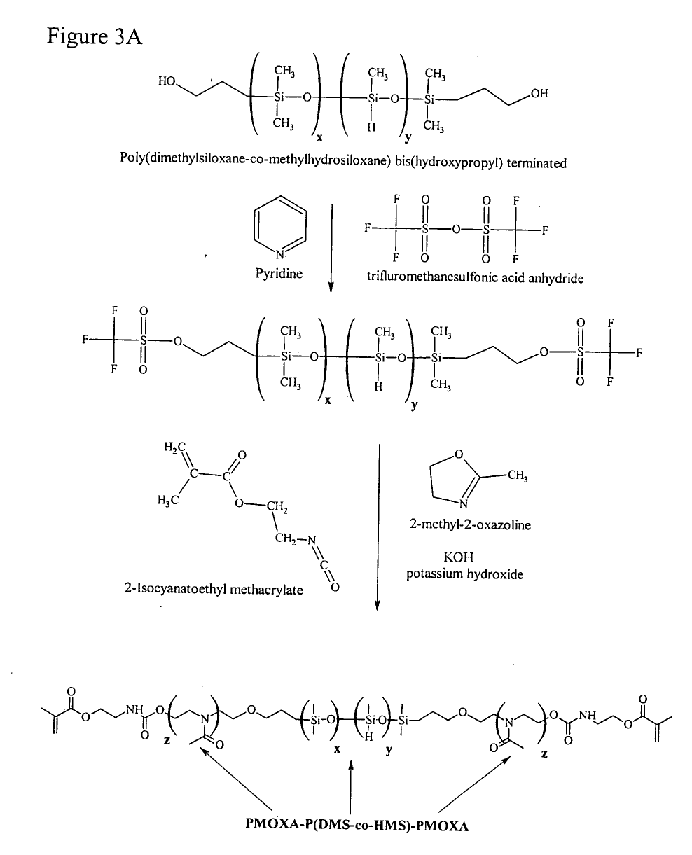

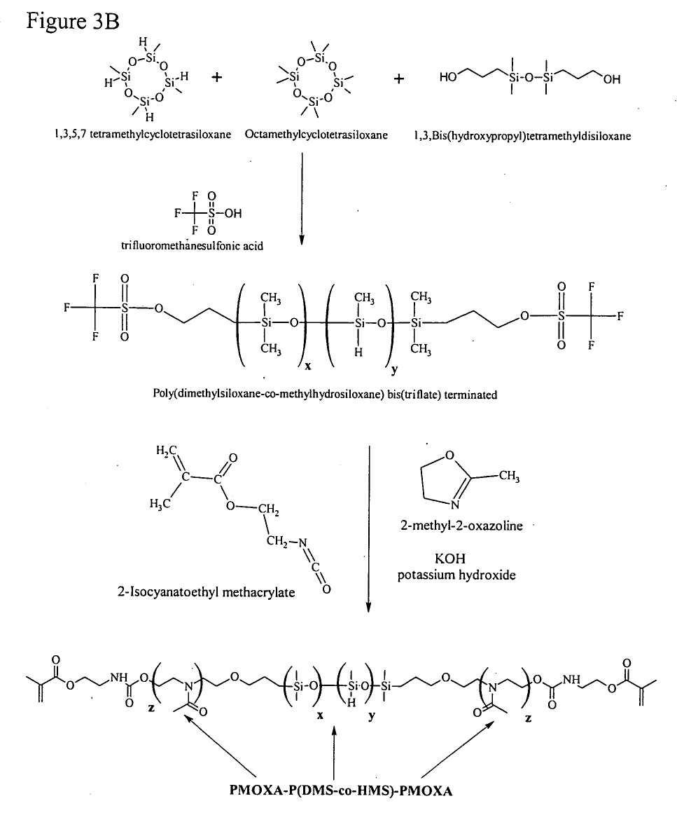

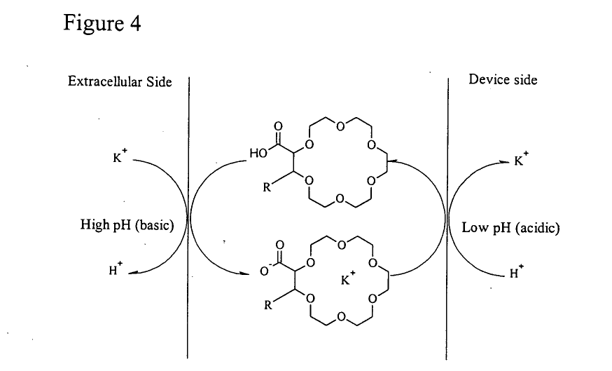

FIG. 3 shows a scheme for synthesis of the triblock polymer. An alternate synthesis scheme is presented in FIG. 4 [7, 53]. Additional preferred ABA polymers include PolyEthylOxazoline-P(DMS-co-MHS)PolyEthylOxazoline, PolyEthyleneOxide-P(DMS-co-MHS)PolyEthyleneOxide, and PolyCaprolactone-P(DMS-co-MHS)PolyCaproLactone. Preferred ABC polymers include PolyEthyleneOxide-P(DMS-co-HMS)PolyOxazoline (Ethyl or Methyl) [61, 62].

Molecules such as molecular carriers can be incorporated into a membrane or attached to its surface using a variety of methods as described in U.S. Pat. No. 6,723,814. For example, a molecular carrier can be incorporated during formation of the membrane, by including it in the polymer solution. The molecular carrier can be covalently or noncovalently associated with the polymer. Specific methods are described herein.

A molecular carrier can also or alternatively be incorporated into the membrane after the membrane has been formed. In one embodiment, a biological molecule is inserted into the membrane after the membrane has been formed by including the molecule in a solution placed on one side of the membrane. Insertion of the molecule into the membrane may be accelerated by applying a potential across the membrane. Molecular carriers can also be incorporated into or onto the membrane in ways other than direct insertion into the membrane. For example, a reactive group on the segmented polymer, such as a methacrylate end group, can be used to react with a reactive group (e.g. an amino or thiol) on a protein, leading to the formation of a covalent bond between the membrane and the protein. As a result the protein would be immobilized at the surface of the membrane rather than within the membrane.

In certain embodiments of the invention the molecular carriers are operably associated directly with the storage/release component itself rather than with a distinct uptake component The molecular carriers may be partially or entirely embedded within the storage/release layer, provided that they are in communication with the environment external to the device from which the stimulating species is to be transported.

C. Carriers and Mechanism of Action

In preferred embodiments of the invention the stimulating species is replenished by sequestering it from the external environment of the device, e.g., extracellular fluid or tissue culture medium. The uptake component accordingly comprises a molecular carrier that selectively permits the stimulating species to enter the device. In a preferred embodiment the molecular carrier complexes with the stimulating species and translocates it into the device up its concentration gradient. Preferably if the stimulating species is an ion, the carrier has at least a 2-fold selectivity for the stimulating species relative to that of other ions, i.e., the ratio of the number of desired ions transported into the device relative to any undesired ion transported into the device is at least 2. More preferably the carrier has at least 5-fold, yet more preferably at least 10-fold selectivity for the stimulating ion. Preferably the carrier has a selectivity for the stimulating species of at least 2-fold, more preferably at least 5-fold, yet more preferably at least 10-fold relative to that of all other ions transported by the molecular carrier combined. Similarly, if the stimulating species is a neurotransmitter, preferably the carrier has at least a 2-fold selectivity for the stimulating species relative to that of other neurotransmitters, i.e., the ratio of the number of desired neurotransmitter molecules transported into the device relative to any undesired neurotransmitter molecules transported into the device is at least 2. More preferably the carrier has at least 5-fold, yet more preferably at least 10-fold selectivity for the stimulating neurotransmitter. Preferably the carrier has a selectivity for the stimulating species of at least 2-fold, more preferably at least 5-fold, yet more preferably at least 10-fold relative to that of all other transported neurotransmitters combined. As mentioned above, K⁺ and dopamine are taken as the representative stimulating species for descriptive purposes.

A large number of K⁺-selective materials are known in the art and are of use in the present invention. Carrier molecules that translocate K⁺ include non-electrogenic simple, symmetric carriers, such as the naturally occurring macrocyclic carrier valinomycin, and various synthetic carriers based on crown ethers or calix[n]arenes, and the nonelectrogenic antiport carriers, which include the naturally occurring acyclic carrier nigericin, synthetic acyclic polyethers, and the macrocyclic lariat crown ethers.

Crown ethers are heterocyclces that, in their simplest form, are cyclic oligomers of dioxane [U.S. Pat. Nos. 3,361,778; 3,987,061; 4,523,994; references 60, 63-67]. The repeating unit of a simple crown ether is ethyleneoxy, i.e., —CH₂CH₂O—. Many crown ethers of use in the present invention comprise at least 4 of these units. Generally ring sizes of macrocycles of use in the present invention range between 9 and about 60. The common names of crown ethers include a number as a prefix to designate the total number of atoms in the ring and a number as a suffix to designate the number of oxygen atoms in the ring [63, 64]. Thus, 15-crown-5 is comprised of 15 atoms in the ring, 5 of which are O and 10 of which are C.

The chemistry of crown ethers typically involves complexation of the ether oxygens with various cationic species, including Ca⁺⁺, Mg⁺⁺, Na⁺, K⁺, Li⁺, etc. This is often termed “host-guest” chemistry, with the ether as host and the ionic species as guest [65]. Host-guest chemistry is also found in a variety of other contexts, e.g., in cyclodextrins and macrocyclic polyether antibiotics. Certain crown ethers display selective complexation with one or more cations. For example, 18-crown-6 ethers are selective for K⁺ while 15-crown-5 ethers are selective for Na⁺. It will be appreciated by one of ordinary skill in the art that a number of variations of the simple crown ether structure exist and are of use in the present invention. For example, substitution of one or more of the O atoms, e.g., by N or S, is common, and the ring can be substituted with a variety of different groups. See, e.g., references [64, 66, 67].

Lariat crown ethers comprise one or more ether side arms that are typically attached to the macroring at nitrogen.

Calix[n]arenes are a class of compounds that have a three-dimensional cavity that can host anions, cations, or neutral species. These molecules are macrocycles that are readily synthesized, e.g., by condensation of p-tert-butylphenol and formaldehyde. The parent p-tert-butylcalix[4]arene adopts a cone conformation possessing a well defined cavity. To alter the properties of the macrocycle, the calixarene scaffold can be modified by functionalization of the methylene groups, and by intraannular and/or extraannular modifications using methods well known in the art. See [60-63] for review and further discussion of these compounds.

U.S. Pub. No. 20040122475 discloses a variety of additional K⁺-selective materials. Materials selective for Ca⁺⁺, Cl⁻, Na⁺ and other ions are also disclosed in U.S. Pub. No. 20040122475 and in U.S. Pat. Nos. 4,214,968, 3,562,129, 3,753,887, and 3,856,649, 4,554,362, 4,523,994, 4,504,368, 4,115,209, 5,804,049, and EP 0 267 724. For example, as mentioned above, various crown ethers are selective for Na⁺. A23187 (Fluka of Buchs, Switzerland), is a commonly used Ca⁺⁺ ionophore. Zeolites are materials that also display selectivity towards various ionic species.

A variety of carriers that transport neurotransmitters are known in the art. For example, lasalocid A is a naturally occurring carboxylic ionophore that performs an uphill transport of dopamine when driven by a pH gradient [56] and can be used as a carrier for dopamine or other biogenic amines. Other suitable carriers include crown ethers comprising boronic acid side groups.

Naturally occurring proteins that function as ion transporters or channels are also of use in the present invention. These proteins generally transfer an ion such as K⁺ across a plasma membrane up its concentration gradient either alone or in combination with another ion such as H⁺ using energy from a proton gradient, ultimately derived from hydrolysis of ATP. Naturally occurring ion transporters or channels, or modified forms thereof, can be produced using recombinant DNA technology, purified from natural sources, etc., and can be embedded in the membrane of the uptake component and optionally covalently attached thereto. See, e.g., Hille, B., Ion Channels of Excitable Membranes (3rd Edition), Sinauer Associates, 2001. Similarly, naturally occurring proteins that function as neurotransmitter transporters are known in the art, e.g., dopamine transporter, serotonin transporter, etc., and can be used in the present invention.

One of the most extensively studied carriers that translocates K⁺ is valinomycin [5], dodecadepsipeptide, where a depsipeptide is a molecule with both peptide and ester bonds. Valinomycin and various synthetic analogues based on bis-crown ethers are neutral carriers and become ionized when a complex is formed with the potassium ion. The translocation can be described by a simple symmetric four state carrier model [6]. The kinetics of the translocation are described by the dissociation or binding constant and the transport rate constant. Since the complex is ionized, application of a voltage will increase the transport rate of the molecule in the favored direction (towards the negative potential) and can be used to move the ion up a concentration gradient. Thus according to certain embodiments of the invention a voltage is applied across the uptake component to drive transport of a stimulating species into the device. However, as the ion is translocated it opposes the applied voltage and decreases the energy available for further transport, resulting in low efficiency of utilization of the applied voltage [7]. Though applied voltage increases the transport rate in one direction over the other, it does not change the binding and unbinding constant of the complex which makes this type of carrier relatively inefficient for transport up a concentration gradient.

Therefore, in preferred embodiments the present invention employs a mechanism in which a concentration gradient of a second species is used to convert the transport of the stimulating species from a process that is thermodynamically unfavorable to one that is favorable. In preferred embodiments the second species is a proton. In certain embodiments energy obtained from light is used to transport the second species into the device. In other embodiments a chemical reaction is used to generate the second species inside the device.

Preferably the carrier can reversibly associate with both the stimulating species and the second species. Thus preferred carriers contain at least one site that reversibly associates with the stimulating species and at least one site that reversibly associates with the second species. By “reversibly associates” is meant that the carrier and a species can associate at a particular time, can subsequently disassociate at a later time, and that this process can be repeated, preferably for at least hundreds of cycles of association and disassociation. It will be appreciated that association is mutual, i.e., the carrier associates with the species, and the species associates with the carrier.

The site for association of the carrier and the stimulating species and the site for association of the carrier and the second species can be the same or different, and there can be more than one site for association with either of the species, or both. The carrier may be able to associate with both the stimulating species and the second species simultaneously, or the association can be mutually exclusive such that at any given time a molecular carrier can be associated with either a molecule of the stimulating species or a molecule of the second species, but not both. It will be appreciated that at any given time individual molecular carriers may or may not be associated with a stimulating species or a second species, i.e., the carriers need not all be in the same state of association or disassociation. The association can be a covalent or noncovalent. In preferred embodiments the association is a “host-guest” association, an ionic association, a hydrogen bond association, or a combination thereof.

In accordance with the invention the carrier transports the stimulating species from the external environment of the device into the storage/release component and transports the second species from the storage/release component into the external environment of the device. The carrier associates with the second species at the interface between the uptake component and the storage/release component and also disassociates with the stimulating species at this interface. In certain embodiments of the invention association of the carrier with the second species destabilizes the complex formed by the carrier and the stimulating species, thereby favoring disassociation of the carrier and the stimulating species. The carrier then transports the second species across the uptake component and releases it at the interface between the uptake component and the storage/release component, where the concentration of the second species is lower than it is in the storage/release component. While present at this interface the carrier associates with the stimulating species. The carrier then transports the stimulating species across the uptake component to the interface between the uptake component and the storage/release component where it releases the stimulating species and associates with the second species once again. This cycle repeats, thereby providing continuous uptake of the stimulating species into the release component. Association of the carrier and the second species at the interface between the uptake component and the storage/release component and disassociation of the carrier and the second species at the interface between the uptake component and the external environment are thermodynamically favorable because the device maintains a higher concentration of the second species in the storage/release component than is present in the external environment. This concentration gradient provides the energy to maintain the cyclical transfer.

In a preferred embodiment the carrier contains a site that can exist primarily in either an ionized or unionized state within a pH range of between about 5.0 to about 9.0, preferably betweeen about 6.0 and 8.0. For example, preferably the second species is a proton, and the carrier contains a site that can exist primarily either in the protonated or unprotonated state within a pH range of between about 5.0 to about 9.0, preferably betweeen about 6.0 and 8.0. A variety of functional groups are known in the art that can exist primarily either in the protonated or unprotonated state within a pH range of between about 5.0 to about 9.0, preferably betweeen about 6.0 and about 8.0. Examples of such ionizable groups include hydroxyl groups (—OH); carboxyl groups (—COOH), amine groups (—NH₂, —NHR, or —NR₂), amides (—(C═O)—NH₂) where R is a carbon-containing moiety, optionally substituted with one or more heteroatoms (e.g., N, S, O, P, B). Molecules containing these groups can undergo reactions such as the following:
R—OH<--->R—O⁻+H⁺
R—COOH<---->R—COO⁻+H⁺
R—NH₃⁺<--->R—NH₂+H⁺

Carriers comprising hydroxyl, carboxyl, or amine functional groups are suitable, e.g., carriers having formula R¹-L-OH, R¹-L-COOH or R¹-L-NH₂, where L is an optional linking moiety and R¹ is a moiety that is capable of reversibly associating with a stimulating species of interest. R¹ can be any of a variety of moieties, e.g., a crown ether, a calix[n]arene, etc. L can be any moiety such as a substituted or unsubstituted aryl or alkyl (which may be saturated or unsaturated), etc., that attaches the ionizable group to R¹. R¹ may comprise a boronic acid side group. In certain embodiments of the invention the stimulating species rather than the carrier undergoes reversible association with the second species. Certain stimulating species such as various biogenic amines are themselves able to undergo reversible protonation and deprotonation and are preferred neurotransmitters for use in the present invention.

In summary, in preferred embodiments of the invention transport of the stimulating species from the external environment of the device (also referred to as the extracellular side) into the storage/release component, is driven by protonation of the carrier or stimulating species at the interface between the uptake component and the storage/release component and deprotonation of the carrier or stimulating species at the interface between the uptake component and the external environment. The carrier and/or the stimulating species comprises an ionizable group. The pH inside the storage/release component is lower than the pH outside the device. A proton gradient thus provides the driving force for repeated cycles of transport accompanied by protonation and deprotonation.

In certain embodiments of the invention the carrier molecules, or a free portion thereof, move back and forth across the uptake component by diffusion. In certain preferred embodiments of the invention the carriers are covalently attached to the bulk material of the uptake component, e.g., to the A, B, or C segments of the ABA or ABC polymer. In this case it will be appreciated that the entire molecule is not free to diffuse. However, the portion of the molecule that contains the sites for reversible association with the stimulating species and second species can preferably still diffuse, e.g., the carrier is attached to the bulk material of the uptake component via a linking portion or tether, such as a hydrocarbon chain. Any of a variety of suitable methods and linkers known in the art for attachment of organic compounds to one another can be used to attach a carrier species to a polymeric material. It may be desirable to increase the concentration of carrier in embodiments in which the carrier is covalently attached to the bulk material of the uptake component.

It will be appreciated that a single carrier molecule need not participate in uptake and discharge at both interfaces. Instead, in certain embodiments of the invention the stimulating species and second species migrate between carrier molecules within the uptake component, eventually reaching the interfaces. This process may be referred to as ion hopping [9, 19, 36]. For example, a first carrier molecule undergoes association with the second species at the interface between the uptake component and the storage/release component and discharges the stimulating species into the storage/release component. The second species is then transferred to a second carrier molecule, which may disassociate from the stimulating species and transfer it to the first carrier. The second carrier may then transfer the second species to a third carrier, which disassociates from the stimulating species with which it is associated and transfers it to the second carrier. The second carrier may then transfer the stimulating species to the first carrier, which has in the meantime discharged the stimulating species with which it was bound into the storage/release component. Similar processes occur simultaneously throughout the uptake component. In this manner molecules of the stimulating species are transferred from one carrier to another and ultimately transported from the interface between the uptake component and the external environment to the interface between the uptake component and the storage/release component. Similarly, the second species is transported in the opposite direction.

The uptake component can comprise multiple different molecular carrier species that all mediate transport of the same stimulating species, or multiple different carrier species that mediate transport of different stimulating species. For example, the uptake component may comprise a carboxylic crown ether that transports K⁺ and a boronic acid crown ether that transports a catecholamine such as dopamine.

The amount of molecular carrier used depends on various factors such as the efficiency with which the carrier transports the stimulating species, the density of molecular carriers, the width of the uptake component, the desired amount of stimulating species to be released, and the expected frequency of release. In general, the molecular carrier(s) make up approximately 0.1%-50% by dry weight of the uptake component. Typically the molecular carrier(s) constitute between approximately 0.5%-45% by dry weight of the uptake component. In certain embodiments the molecular carrier(s) constitute between 0.25%-30% by dry weight of the uptake component.

Nigericin is a naturally occuring antibiotic carrier that meets the above criteria, e.g., it exchanges K⁺ for H⁺ by undergoing reversible association with K⁺ and H⁺[19-21]. In certain embodiments of the invention nigericin or a related compound is used as the carrier for K⁺ ions.

In a particularly preferred embodiment of the invention the carrier molecule is a carboxylic acid crown ether [25-27]. Carboxylic acid crown ethers represent a preferred class of compounds that are able to pump alkali metal ions up their concentration gradients [29-33]. These molecules are crown ethers that have at least one carboxyl moiety attached thereto, e.g., to a carbon in the ring. The carboxy moiety may either be directly attached to the ring or may be attached via a linking moiety L, e.g., a saturated or unsaturated, substituted or unsubstituted, hydrocarbon chain. Carboxylic acid crown ethers may have one or more groups R attached to the ring. R can be, e.g., an aryl or a saturated or unsaturated alkyl moiety, either of which can be substituted or unsubstituted and may contain one or more heteroatoms. In certain embodiments R is a relatively long alkyl chain (e.g., at least 8-20 carbons in length), which increases the lipophilicity of the crown ether carboxylic acids and enhances its partitioning into the hydrophobic phase of the uptake component membrane. R can comprise one or more carboxy or hydroxyl groups.

FIG. 4 shows the mechanism of coupled countertransport of K⁺ and protons by a carboxylic acid crown ether and is representative of the mechanism by which the neural stimulation device accumulates a stimulating species from the external environment of the device in preferred embodiments of the invention. As shown in FIG. 4, when a proton gradient is present the carboxylic acid crown ether deprotonates at high pH (low [H⁺]) and will become negatively charged. The crown ether has the appropriate cavity size (in this case it is a 18-crown-6-ether) for complexing a K⁺ ion at the interface between the uptake component and the external environment (extracellular side). Since there is a counterion on the carrier (the negatively charged oxygen atom) it will be favorable to form a complex with the positively charged cation. Having formed a complex, the carrier diffuses towards the side with the higher concentration of protons, where it will re-protonate. This makes the potassium-crown ether complex unstable and hence it will disassociate, giving up the cation (K⁺) to the low pH (high [H⁺]) side. The cycle would repeat until the concentration gradients of [H⁺] and [K⁺] are equal. However, according to the invention protons are transported into the storage/release component using energy derived from light, or protons are generated within the storage/release component. Therefore, the cycle continues until a dynamic equilibrium is reached in which a minute flux that is equal to the discharge rate of the stimulating species through the release component is maintained. This flux can be tailored using both the geometry and the chemical composition of the release layer as tuning parameters. This could also be used as a self regulatory mechanism where the concentration of the stimulation species inside the device does not exceed a certain value.

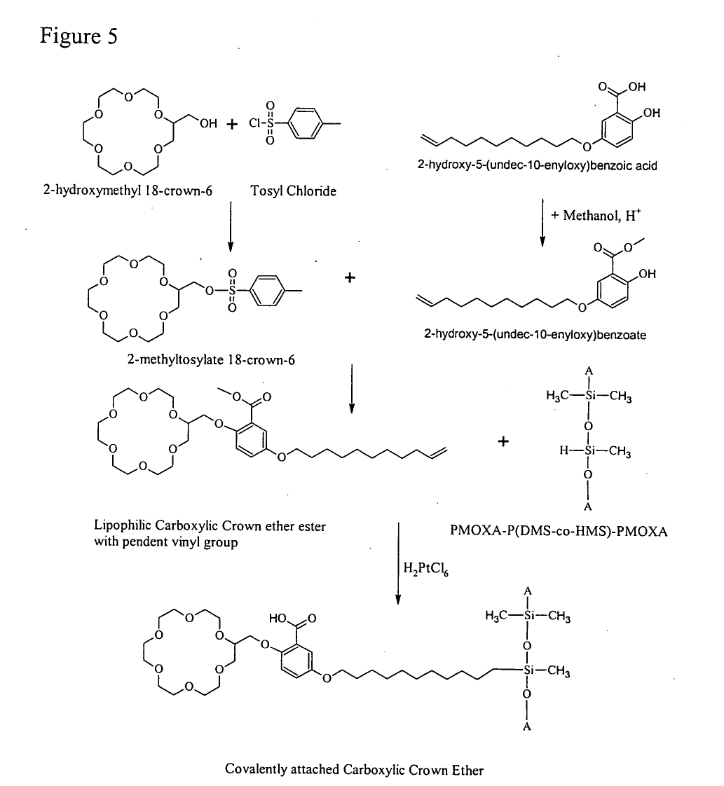

In a preferred embodiment the carboxylic acid crown ether is covalently attached to a hydrophobic B segment of an ABA or ABC polymer, e.g., to the B segment of PMOXA-P(DMS-co-HMS)-PMOXA. FIG. 5 shows an exemplary synthetic scheme for synthesis of a crown ether carboxylic acid having a pendant vinyl group [35]. Attachment of the carboxylic acid crown either to the polymer is achieved via a pendant vinyl group [17, 34]. It will be appreciated that this scheme could be used for to synthesize a wide variety of proton ionizable crown ethers, e.g., carboxylic acid crown ethers, and could be used for attaching a crown ether to other ABA or ABC polymers, particularly those in which the B segment comprises P(DMS-co-HMS). The invention therefore provides a PMOXA-P(DMS-co-HMS)-PMOXA polymer having a crown ether covalently attached thereto. In certain embodiments of the invention the crown ether is a substituted crown ether, e.g., a carboxylic acid crown ether or a crown ether comprising a boronic acid group.

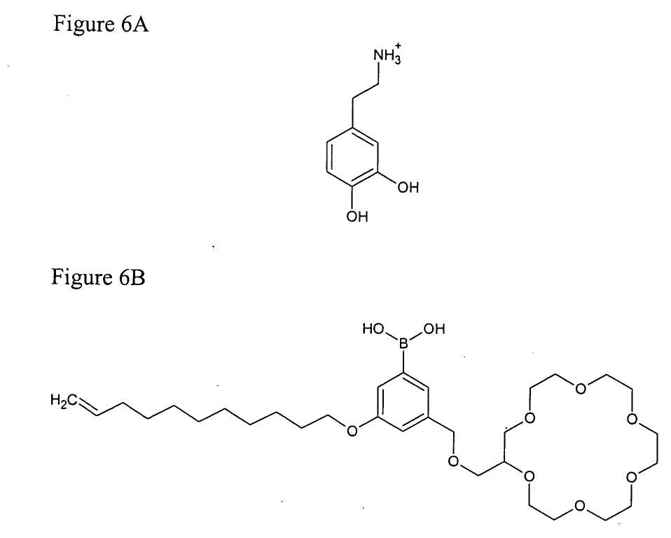

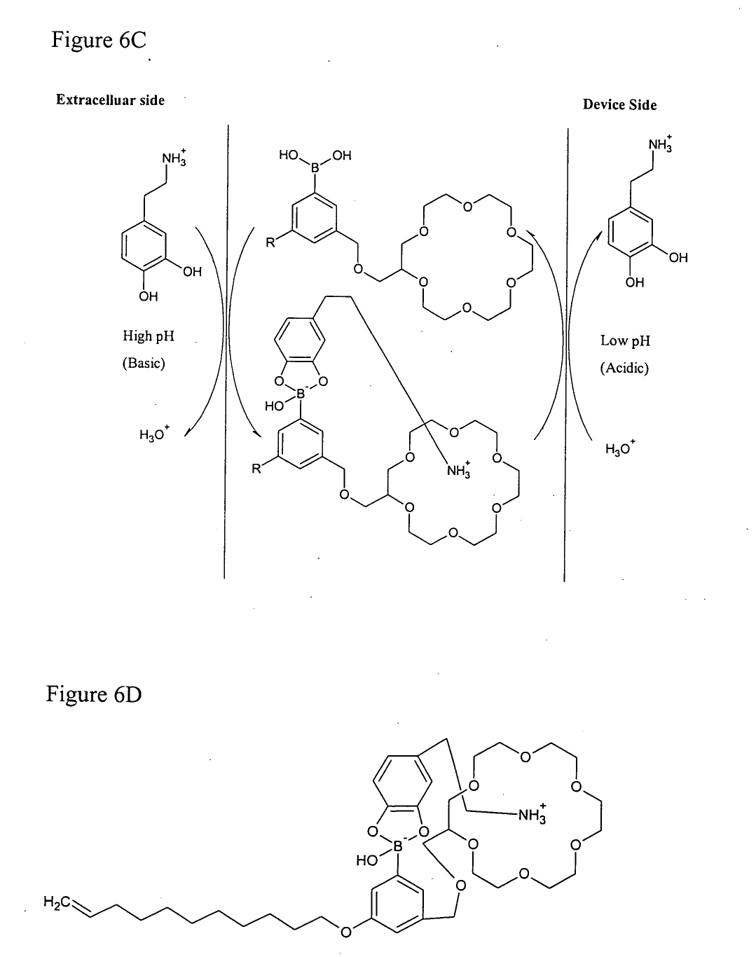

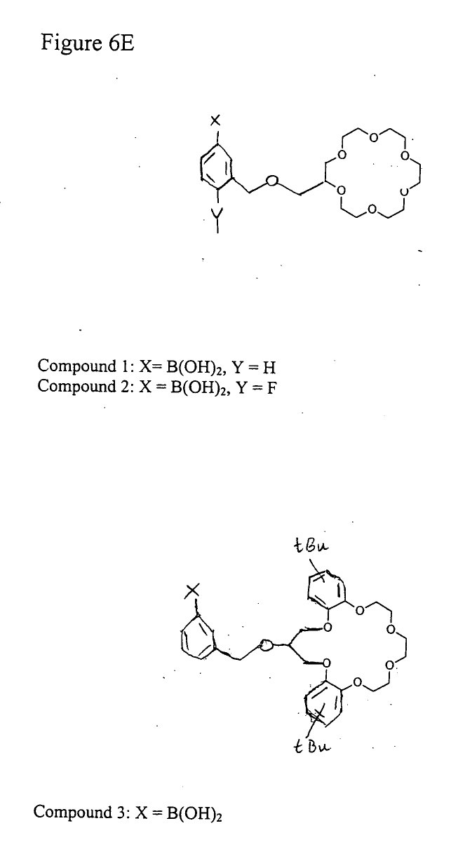

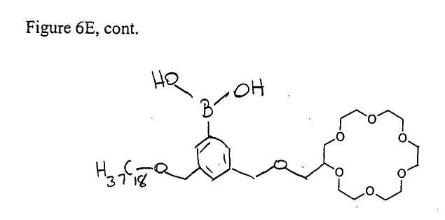

In another preferred embodiment a crown ether comprising a boronic acid side group is used to transport a biogenic amine neurotransmitter, e.g., a catecholamine, into the storage/release component. Crown ether boronic acids show pH driven transport of dopamine and other catecholamines [57, 58]. Dopamine (FIG. 6A) is used as an exemplary biogenic amine, but other neurotransmitters can also be transported using this carrier.

FIG. 6B shows a crown ether comprising a boronic acid side chain that can be used to transport catecholamines. The lipophilic carrier comprises a pendant vinyl group for covalent attachment to the bulk material of the uptake component. Synthesis of the carrier shown in FIG. 6B is performed as described [57, 58]. The pKa of the boronic acid containing carrier is approximately 9, and the carrier is uncharged in the source phase (i.e., the external environment of the device), where the pH is around 7. FIG. 6C is a schematic diagram showing proton-coupled transport of dopamine. Condensation of the boronic acid with the diol motif of the catecholamine generates the boronate ester which is much more acidic than parent boronic acid and carriers a formal negative charge. The zwitterionic species shown in FIG. 6D can now be transported though the membrane phase. On reaching the interface with the storage/release component (device side), where the pH is low, the catecholamine dissociates and regenerate the boronic acid carrier, which is now available for the next cycle. Covalent attachment of the boronic acid containing crown ether via the pendant vinyl group proceeds as described above for carboxylic acid crown ethers. FIG. 6E presents the structure of other catecholamine transporters that could be used. A number of other synthetic ditopic catecholamine transporters are known in the art and are of use in the practice of the present invention. The transporters may be used individually or in combination.

Carriers that perform proton-coupled transport of a variety of other amines, e.g., tryptophan, glutamate, serotonin, etc., are known in the art and are of use in the present invention [71-73]. In some embodiments of the invention a precursor of a neurotransmitter is transported into the device, and the precursor is converted into the active stimulating species by, e.g., an enzyme immobilized within the device.

It is noted that the compounds described herein may be produced using a variety of methods, some of which are described above. For purposes of the present invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. It will be appreciated as described below, that a variety of compounds can be synthesized according to the methods described herein. In general, the starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1989, “Comprehensive Organic Transformations”, VCH Publishers. These schemes are merely illustrative of some methods by which the compounds described herein can be synthesized, and various modifications to these schemes can be made and will be suggested to a person of ordinary skill in the art having regard to this disclosure.

Furthermore, it will be appreciated by one of ordinary skill in the art that the synthetic methods, as described herein, utilize a variety of protecting groups. By the term “protecting group”, it is meant that a particular functional moiety, (e.g., amine, hydroxyl, carboxylic acid, ketone, aldehyde, thiol, imine) or atom, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

D. Storage/Release Component

In a preferred embodiment of the invention a single component serves both as a storage and release component and will be referred to as a “storage/release component”. The storage/release component should have sufficient volume to sequester enough of the stimulating species to allow for release of at least enough of the species to stimulate a nearby neuron or neural target cell. The storage/release component should be able to respond to an input signal to release the stimulating species into the external environment of the device.

As mentioned above, the storage/release component should be in physical communication with the uptake component so that the stimulating species can be transferred into the storage component. Thus there is an interface between the storage/release component and the uptake component. There is also an interface between the storage/release component and the external environment, so that the storage/release component can release the stimulating species in the vicinity of a neuron or neural target cell. If the storage and release functions are performed by individual components, these components should be in physical communication so that transfer of the stimulating species from the storage component to the release component can be achieved. It will be appreciated that the device can assume a variety of different configurations and that there can be multiple distinct compartments for uptake, storage, and/or release.

In a preferred embodiment of the invention the storage/release component comprises a stimulus-responsive hydrogel. In general, a hydrogel is a polymeric network capable of imbibing and retaining large quantities of water without dissolution or loss of its three-dimensional network structure. Stimulus-responsive hydrogels are materials whose properties change in response to a range of environmental stimuli. The property that typically changes in the most dramatic manner is volume. The change may occur discontinuously at a specific stimulus level or gradually over a range of stimulus levels. Hydrogels that alter their volume in response to any of a variety of stimuli are known in the art [12, 42, 45, 46, 48 and references in the foregoing]. In preferred embodiments of the invention the hydrogel responds to a stimulus by reducing its volume. The alteration in volume (collapse) results in release of the stimulating species. The volume is restored in a short period of time following release.

Preferred stimulus-responsive hydrogels for use in the present invention respond to a change in pH. pH sensitive hydrogels undergo a very large and reversible volume change in response to pH changes within the hydrogel. The pH sensitivity is typically caused by pendant acidic and basic groups, e.g., carboxylic acids, sulfonic acids, primary amines, and quaternary ammonium salts. The critical pH value at which the pH-sensitive hydrogel undergoes a volume transition can be controlled by selection of pendant groups with the appropriate pKₐ values and by adjusting the hydrophobicity by choosing among a nuber of monomers, e.g., poly(alkyl acrylate), poly(alkyl methacrylate), poly(2-hydroxyethyl methacrylate) (p-HEMA), poly(2-hydroxypropyl methacrylate) (p-HPMA), poly(acrylamide), poly(N-vinylpyrrolidone), poly(vinyl alcohol), poly(ethylene oxide), and poly(etherurethane). These monomers can be used alone or in various combinations to form copolymers. A variety of different crosslinkers can be used.

In preferred embodiments of the invention the storage/release component comprises a pH-responsive p-HEMA hydrogel [12, 45, 46]. The phase transition pH is preferably tailored to be close to physiological pH (e.g., preferably between about 6.5 to 8.5). p-HEMA can be patterned by photolithography, making it amenable to conventional microfabrication [47, 48]. p-HEMA contains the same polymerizable functional groups as the end groups of the PMOXA-P(DMS-co-HMS)-PMOXA membrane which is used as the bulk material of the uptake component in a preferred embodiment of the invention. This helps to make the attachment of the membrane to the gel layer mechanically robust.

The kinetics of the gel, e.g., the speed with which it responds to a change in pH, are tailored to obtain the time constants of interest, e.g., ˜30 msecs, which is approximately the frame rate of the eye, for a retinal prosthesis. The time constant is tailored by introducing graft chains in the gel layer and/or by using surfactants which form water channels [13], which allows for faster incorporation or release of water from the gel. It will be appreciated that the small dimensions of the device allow for short time constants, since the time constant is proportional to the square of the thickness. A variety of other stimulus-responsive hydrogels could also be used. In order to achieve release, it will generally be desirable to achieve a change of about 0.7-1.0 pH units, though smaller or larger values could also be used. Thus in certain embodiments the pH inside the device is lowered from the normal body pH by about 10-fold in order to achieve release, e.g., from 7.5 to 6.5 pH units. The change in pH occurs upon receipt of an activating input by the device. The activating input may be, e.g., light or an electrical signal. Receipt of the activating input is coupled to a change in pH in the hydrogel as described below, i.e., an activating input results either directly or indirectly in a change in pH in the hydrogel, resulting in release of the stimulating species.

In certain embodiments of the invention a volume of 100 picoliters (pL) is released, containing approximately 10¹²-10¹³ potassium ions. Smaller or larger volumes, e.g., 5-10 pL up to 100 pL or up to several hundred pL could also be used, with a corresponding change in the number of potassium ions so as to achieve a desired change in potassium concentration (i.e., increase in background potassium concentration). The change in potassium concentration may, in general, range between about 5-10 mM, but greater changes in concentration will provide greater stimulation. Thus for certain applications it may be desirable to release sufficient potassium to cause a concentration change of greater than 10 mM, e.g., 10-15 mM, 15-20 mM, 20-25 mM, 25-50 mM, or even greater in the vicinity of a neuron or neural target cell.

In other embodiments of the invention a hydrogel that is responsive to an applied electric field is used [68-70]. Examples include poly(2-acrylamido-2-methylpropane sulfonic acid)/hyaluronic acid polymer gels [1, 2]. Other electric field sensitive polymers such as N-isopropylacrylamide (NIPAM) gels can also be used [70]. In certain embodiments of the invention an electronic chip receives power and data through an inductively coupled coil system; the chip then sends electrical signals to the neural stimulation device or to an array comprising a plurality of devices, which serves to collapse the gel by the applying an electric field, releasing the stimulating species (FIG. 1F).

While use of a stimulus-responsive hydrogel is a convenient method for achieving both storage and release, other methods are also envisioned. For example, the stimulating species can be transferred into a storage component that is a hollow reservoir equipped with a pump or other fluid-directing means operably connected to the reservoir and effective to cause fluid flow to an opening or channel in fluid communication with the external environment of the device. A pump or fluid-directing means may cause fluid flow by creating a pressure differential, an osmotic differential, or may cause flow by electrical means, e.g., electro-osmotic means, or in other ways. Fluid flow can be achieved using a mechanical pump mechanism, e.g., piezoelectric, pneumatic, peristaltic, electrostatic, or electromagnetic pump. Instead of, or in addition to such mechanisms, fluid flow can be achieved using thermal, chemical, osmotic, acoustic, magnetic, electric, or electrosomotic, means or mechanisms. Suitable pumps include those commonly used in various microelectromechanical systems (MEMS). Examples are discussed, e.g., in U.S. Pat. No. 5,734,395 and in Andersson et al., Sensors and Actuators B 72:259-265 (2001); Morf et al., Sensors and Actuators B 72:266-272 (2001); Morf et al., Sensors and Actuators B 72:273-282 (2001); and Zeng et al., Sensors and Actuators B 82:209-212 (2002). The storage reservoir and release component may be connected, e.g., via microfluidic channels. Suitable materials and methods are described, e.g., in U.S. Pub. No. 20030032946. FIG. 1B shows an embodiment comprising a storage component 30 and multiple release components 36 separated from one another by semi-permeable or permeable membranes. Pumps 38 control release of the stimulating species from individual release components. In this embodiment the storage and release components may be fluid-filled rather than comprising a hydrogel.

E. Means for Producing a Concentration Gradient

As discussed above, in preferred embodiments the neural stimulation device accumulates the stimulating species from its external environment by coupling transport of the stimulating species up its concentration gradient to transport of a second species, e.g., a proton, down its concentration gradient. Therefore in preferred embodiments the device comprises means for producing a concentration gradient of the second species, such that the concentration of the second species is higher inside the device (e.g., in the storage/release component) than outside the device. Preferably the second species is a proton.

In preferred embodiments of the invention a chemical reaction is used to generate protons inside the device, e.g., in the storage/release component. Preferably the device tranports a substrate for the chemical reaction into the device from its external environment. In certain embodiments of the invention protons are generated inside the device by the oxidation of glucose into gluconic acid using glucose oxidase [39], a naturally occurring enzyme that is used in many amperometric in vivo glucose sensors. Glucose oxidase (classified under heading 1.1.3.4 according to the nomenclature recommendations of the Nomenclature Committee of the International Union of Biochemistry and Molecular Biology) is an enzyme that catalyzes the following overall reaction:
glucose+O₂+H₂O—→gluconic acid

Gluconic acid freely dissociates (pKa˜3.6) into gluconate [40, 41, 42] giving up a H⁺. Additionally another enzyme, catalase, may be used to decompose hydrogen peroxide into water to prevent glucose oxidase from peroxide induced degradation[41]. The overall reaction may be then given by

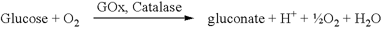

Thus the above reaction produces protons which are used to drive uptake of the stimulating species. Glucose is a preferred substrate for a proton-producing chemical reaction since the body naturally maintains glucose levels that provide sufficient amounts for operation of the device.

Carbonic anhydrases (classified under heading 4.2.1.1 according to the nomenclature recommendations of the Nomenclature Committee of the International Union of Biochemistry and Molecular Biology) are enzymes that catalyze the hydration of carbon dioxide and the dehydration of bicarbonate:
CO₂+H₂O<----->HCO₃⁻+H⁺

Carbonic anhydrase enzymes are metalloenzymes consisting of a single polypeptide chain (Mr˜29,000) complexed to an atom of zinc. They are widespread in nature, being found in animals, plants, and certain bacteria. In animals they play an important role in respiration by facilitating transport of CO₂ and are involved in the transfer and accumulation of H⁺ and HCO₃⁻. Carbonic anhydrases are extensively reviewed in 55. The reaction catalyzed by carbonic anhydrase can be used to generate protons inside the device. Carbon dioxide is a product of cellular respiration and is therefore available as a substrate and can diffuse into the hydrogel down its concentration gradient as it is consumed in the reaction. Bicarbonate can diffuse out of the device down its concentration gradient as it is produced.

Glucose oxidase and catalase can be purified from natural sources or produced using recombinant DNA technology. Both enzymes are available commercially, e.g., from Worthington Biochemical Corporation, Lakewood, N.J., 08701. Preferably the enzyme that catalyzes the proton-producing reaction is immobilized in the bulk material of the storage/release component, e.g., a p-HEMA storage layer, using established techniques [40, 42, 44].

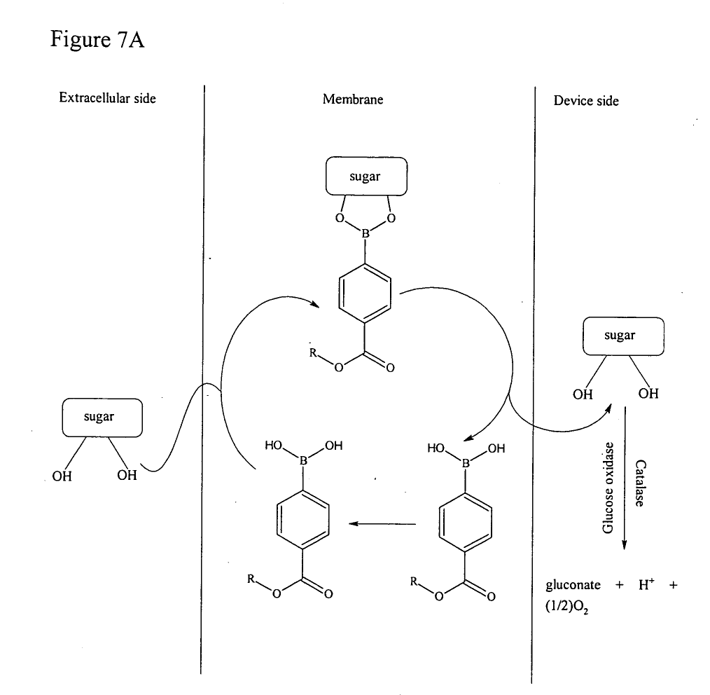

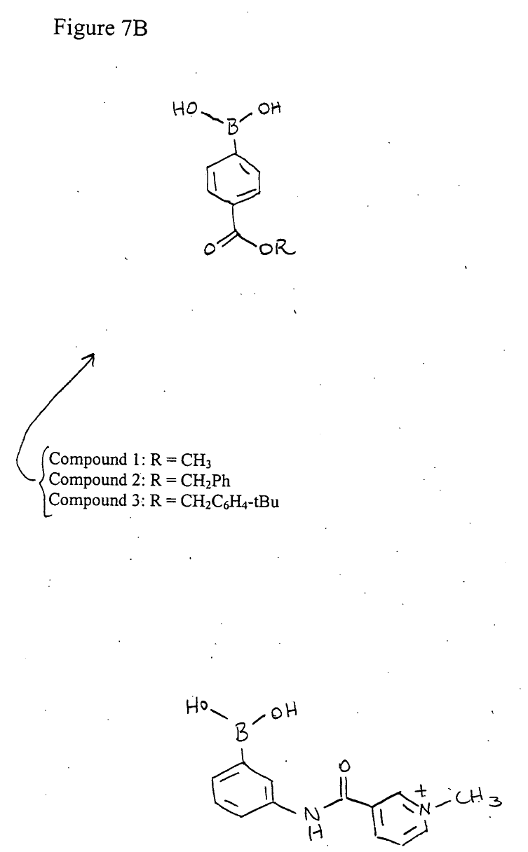

In order to provide a substrate for glucose oxidase, glucose needs to be transported into the device, i.e. the device should be permeable to glucose. In certain embodiments of the invention synthetic glucose carriers based on boronic acids are used [43]. While not wishing to be bound by any theory, the likely mechanism of glucose transport is shown in FIG. 7A. The sugar is complexed by the boronic acid moiety and passively transported from one side to the other. Since there is an active consumption of glucose inside the device, there is a steady flux of glucose into the device due to the finite concentration difference between the inside and outside of the device. A variety of different boronic acids, having the structure RB(OH)₂, may be used as glucose carriers. R can be, e.g., an aryl or a saturated or unsaturated alkyl moiety, either of which can be substituted or unsubstituted and can contain one or more heteroatoms (e.g., N, S, O, P, B, F, Br). Exemplary boronic acid carriers are described in [43]. In certain embodiments a boronic ester is used. These are compounds of the formula —B(OR)₂ wherein R is typically an alkyl group. Under aqueous conditions, many boronic esters hydrolyze to form boronic acid. Therefore, OR groups that hydrolyze to OH are of use in the present invention. The two R groups may be linked to form a cyclic structure (e.g., —CH2-CH2-). Additional boronic acid glucose transporters include (3,5-dichlorophenyl)boronic acid, [3,5-bis(trifluoromethyl)phenyl]boronic acid, (4-bromophenyl) boronic acid, etc. Further examples are shown in FIG. 7B.

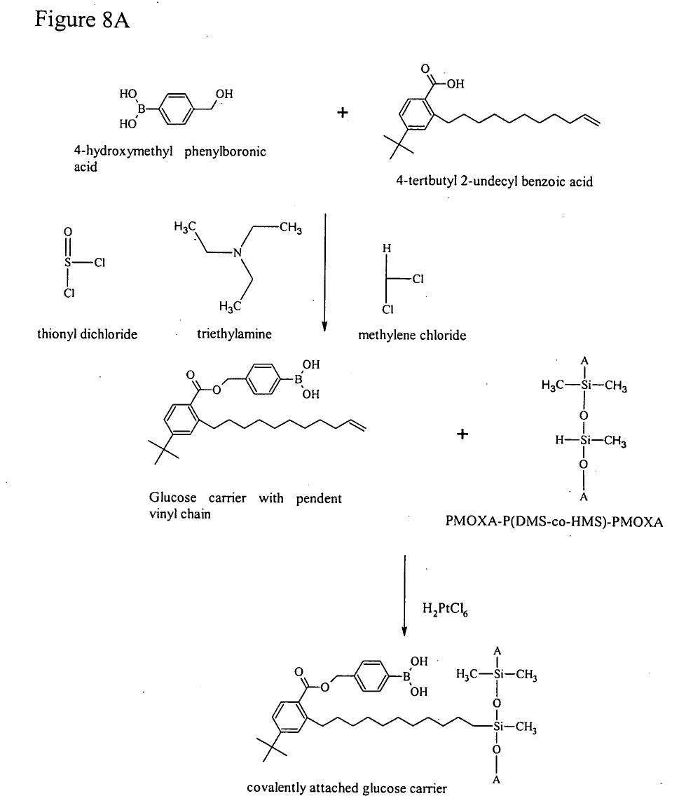

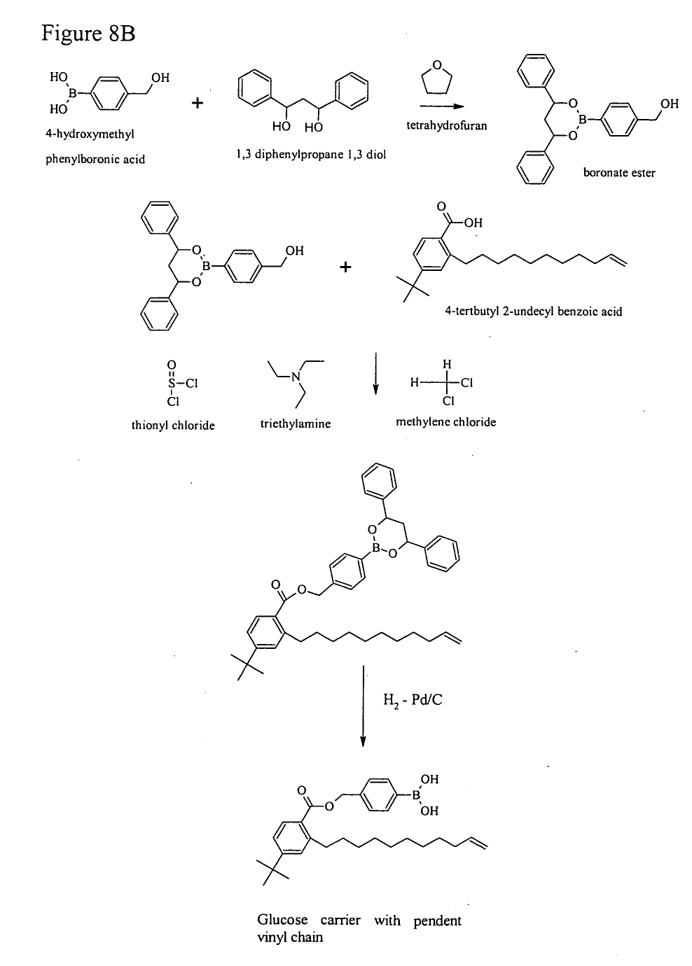

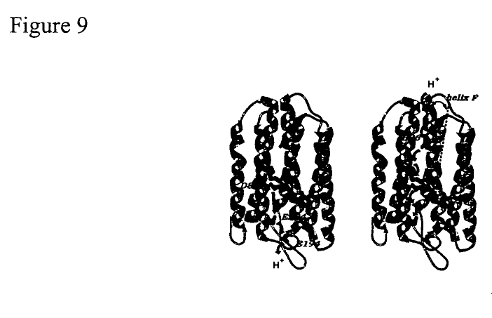

The glucose carriers are preferably covalently attached to the uptake component to prevent leaching of the carrier. In a preferred embodiment the glucose carriers are covalently attached to the siloxane layer (B segment) of an ABA polymer. A suitable attachment procedure is shown in FIG. 8A. The boronic acid can also be provided with protecting groups for the synthesis, as shown in FIG. 8B. The synthesis may also be done with a 1,2 benzene dimethanol or pinacol [79] instead of 1,3 diphenyl propane 1,3 diol as the protecting group for the boronic acid.

While producing a proton gradient by means of a chemical reaction that generates protons is preferred, other methods for generating a proton gradient can also be used. For example, proton pumps found in the photoreaction centers of a variety of bacteria can be used, e.g., proton pumps such as bacteriorhodopsin from the bacterium Halobium Halobacteria, or the photoreaction center of Rhodopseudomonas viridis, which also acts as a light driven proton pump [39] and responds to infrared light could be used. Use of the photoreaction center of Rhodopseudomonas viridis to transport protons into the storage/release component for purposes of producing a proton gradient may be preferable as this would allow the use of bR for responding to light in the context of a visual prosthesis. Thin films comprising such proton pumps can be attached to a surface of the storage/release component, or alternately proton pumps may be embedded in the surface. In preferred embodiments of the invention proton pumps are reserved for use as light-sensitive elements that trigger release of the stimulating species as described further below. It will be appreciated that energy for the transport of the stimulating species may be provided by creating a gradient of ions or compounds other than protons, though use of a proton gradient as described herein is generally preferred.

F. Receiving an Activating Input and Coupling it to Release of the Stimulating Species

To function effectively, the neural stimulation device should comprise a means of receiving an activating stimulus and of coupling receipt of the activating stimulus to release of the stimulating species. Accordingly, as shown in FIGS. 1A-1F, neural stimulation device 10 comprises a release trigger 90. The activating input may be e.g., light, a chemical signal, or an electrical signal. The device may respond directly to a typical stimulus in the environment of a subject (e.g., light, a chemical, motion, etc.) or additional means of sensing the environmental stimulus and transducing it to provide an activating input to the device may be employed. Alternately, activating inputs to the device may be artificially generated, e.g., using a computer. Thus in various embodiments receipt of an activating input and release of the stimulating species are accomplished without use of an electronic components, while in other embodiments the device comprises any of a variety of electronic components.

A. Biochemical Implementation

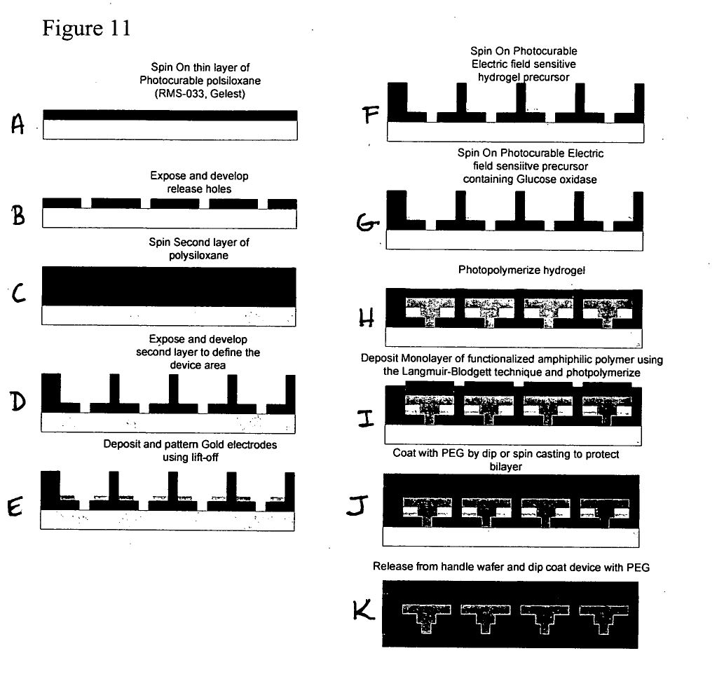

In certain embodiments the release trigger comprises a light-sensitive element. In certain embodiments of the invention the light-sensitive element is a light driven proton pump 92 that is used to locally decrease the pH in the storage/release component. Conversion of light into proton gradients is a widespread mechanism found in many photosynthetic bacteria. Such bacteria contain proteins that act as proton pumps in response to absorption of a photon. For example, bacteriorhodopsin (bR) is the sole protein found in the membrane of the salt loving bacteria, Halobium Halobacteria [11, 37, 49]. This protein is very similar to the protein found in the visual pigment of the eye, rhodopsin [50]. Furthermore, it is widely studied and commercially available. Bacteriorhodopsin films are commercially available and can be attached to polymers such as PDMS using, for example, layer by layer assembly.
bR is a seven-helical transmembrane protein that contains retinal (vitamin A aldehyde) as its chromophore (light sensitive material). It is attached to a lysine molecule by means of a Schiff's base. In its natural environment of the cell membrane, the photo-cycle is initiated when retinal is photoisomerized from an all-trans to a 13-cis configuration. This rapid rearrangement of the electronic structure of retinal due to photon absorption reduces the proton affinity of the charged Schiff base nitrogen which loses its proton (deprotonates), while the initial acceptor group, Asp85, protonates. Simultaneously with the protonation of Asp85, a proton is released at the extracellular surface of the protein. Subsequently, Schiff base reprotonation takes place from the cytoplasmic side. In response to large conformational rearrangements, Asp96, which is protonated in the ground state, passes its proton to the deprotonated Schiff base 11 Å away (N intermediate in FIG. 11). Asp96 is subsequently reprotonated from the cytoplasmic side. To complete the cycle, the retinal needs to re-isomerize to all-trans and the proton stored on Asp85 moves via waters and Arg82 to reprotonate the terminal proton release group [49].

The proton pumps are either embedded in the storage/release component or provided as a thin film or membrane attached to a surface of the storage/release component. The proton pumps are oriented appropriately so that they pump protons into the device upon absorbing a photon. When light strikes the device, the proton pumps translocate protons into the storage/release component, resulting in a local decrease in pH. The gel then contracts, causing release of the stimulating species from the opening, which is located in the vicinity of a neuron or neural target cell.

FIG. 1C shows a detailed schematic of a preferred embodiment of the invention. The device 10 comprises uptake component 20, which is a polymeric lipid-like membrane as described above having hydrophilic outer layers and a hydrophobic inner layer. Uptake component 20 comprises carriers 24, which are synthetic antiports that transport a stimulating species 40 (in this case K⁺ or a catecholamine) across the uptake component and into storage/release component 30 and countertransports protons 50 out of the device into the external environment of the device. Uptake component 30 further comprises carriers 28, which are synthetic glucose carriers that transport glucose into the storage/release component. Enzyme 80, in this case glucose oxidase, is immobilized in storage/release component 30, preferably close to the interface 32 between the uptake component and the storage/release component. Storage/release component 30 comprises a pH-sensitive hydrogel. Release trigger 90 comprises light-driven proton pumps 92 that transport protons into the device in response to light (hv), causing a decrease in pH in the hydrogel. Stimulating species 40 is then released from the storage/release component via aperture 34. The storage/release component and side walls of the uptake component are encapsulated in polymer layer 100 which may be, e.g., parylene, polysiloxane, etc.

FIG. 1D shows an array 510 comprising a plurality of individual neural stimulation devices 10, as discussed further below.

B. Hybrid Biochemical/Electronic Device

The device described above both senses a stimulus, e.g., light, and releases the stimulating species using biochemical and physical mechanisms but does not require or interface with electronic components. In other embodiments the device comprises any of a variety of electronic components or other ancillary components and may be referred to as a hybrid biochemical/electronic device. The uptake component, storage/release component, and mechanism of uptake of the stimulating species are as described above. However, in certain of these embodiments, as shown in FIG. 1E, release trigger 90 comprises an electrode. The electrode receives electrical signals that are generated in a variety of ways. The electrode is coupled via a communication link 310 to electronic circuitry 300 that typically comprises microprocessor 320, which performs signal processing and control functions. The communication link may be a physical link, e.g., a wire (e.g., a fiber-optic link), or a wireless link. Wireless communication links may be, e.g., infrared links, radio frequency (RF) links, etc. Microprocessor 310 may receive inputs from a stimulus-sensing device 400 via link 410, which may be a physical or wireless link. The stimulus-sensing device may be, e.g., a camera, microphone, pressure transducer, etc. The stimulus-sensing device receives a signal from the environment of a subject. The signal is transformed into an electrical signal that is processed by microprocessor 320, which then sends appropriate electrical signals to release trigger 90 to cause release of the stimulating species. A power source (not shown) is typically provided for data processing, generation of the electronic signals, etc. Power may be supplied through wireless means.

For use as a visual prosthesis, a light-sensitive element such as a camera is employed in certain embodiments of the invention. Suitable cameras for perceiving a visual image and converting it into electrical signals suitable for stimulating a retina are known in the art. See, e.g., U.S. Pat. No. 5,935,155 and U.S. Pub. No. 20030158588. Rather than directly electrically stimulating the retina via an electrode array, the electrical signals are modified appropriately to achieve release of a stimulating species. Image acquisition devices such as CCD cameras, CMOS cameras, video cameras, etc., can be used. In certain embodiments a digital camera is used.

In other embodiments of a visual prosthesis, release trigger 90 comprises a light-sensitive element 92 and an electrode, or light-sensitive elements are appropriately positioned elsewhere in the device so that an incoming light stimulus can be sensed. The light-sensitive element may be, e.g., a photodiode, a bR thin film, etc. The light-sensitive element transduces light into an electrical signal that is transmitted to electronic circuitry 300. Electronic circuitry 300 processes the signal and transmits appropriate electrical signals to release trigger 92 to cause release of the stimulating species.

In other embodiments of the invention the device is used for stimulation of cells outside the visual system. For example, the device can be used for stimulation of the auditory pathway, spinal cord, nerves to the diaphragm, nerves to the bowel, nerves to the bladder, etc., for stimulation of particular muscles, for stimulation of structures in the brain, etc. An appropriate stimulus-sensing device is selected depending upon the particular neurons or neural target cells to be stimulated. For example, a microphone or other sound-sensing device can be used for auditory stimulation. Sound is transformed into an electrical signal, and electronic circuitry 300 processes the signal and generates outputs appropriate to cause release of the stimulating species by the release trigger

In other embodiments of the invention the electrical signals sent to the release trigger are computer-generated, e.g., in response to inputs from a user. For example, a predefined sequence of electrical signals may be used to stimulate muscles, e.g., for purposes of pain relief or to achieve movement, or to cause release of a stimulating species at predefined times for therapeutic purposes.

H. Device Dimensions and Array Configurations

In certain embodiments a plurality of individual neural stimulation devices are assembled into an array, e.g., as shown schematically in FIG. 1D. Each device constitutes an element of the array. The array can include multiple neural stimulation devices that release the same stimulating species or devices that release different stimulating species. This can be accomplished, for example, by attaching or implanting discrete elements in a biocompatible material such as polydimethylsiloxane (PDMS). Alternately, an integrated manufacturing process can be employed as described below. It will be appreciated that those regions of the device that are to remain in contact with the extracellular space (e.g., at least a portion of the outer side of the functionalized uptake component and at least a portion of the exterior surface of the storage/release layer) should not be obscured by the biocompatible material.

The dimensions of each discrete element can vary and can be tailored for different applications. Exemplary dimensions can be, for example, 100 microns×100 microns in horizontal dimensions with a thickness (vertical dimension) of 10-20 microns Horizontal and vertical dimensions are defined assuming the device is oriented as shown in FIG. 1C. The thickness of the uptake component is typically approximately 10 nm. Thus most of the thickness consists of the storage/release layer. Other exemplary dimensions are 25 microns×25 microns×5 microns (thickness), or 10 microns×10 microns×5-10 microns (thickness). It is noted that the portion of the storage-release layer that is in contact with the exterior can be much smaller, e.g., 10 microns×10 microns.

Preferably those surfaces of the device that do not need to contact the external environment are encapsulated in a suitable biocompatible polymer such as parylene. In certain embodiments of the invention the surface of the uptake component is coated with a material such as polyethylene glycol (PEG), which dissolves in the body.

Certain embodiments of a visual prosthesis comprise an array that functions both as a release array and as a light-sensitive array. For example, if the release trigger comprises bR, the release trigger both senses light and triggers release of the stimulating species. In other embodiments a separate light-sensitive array is located within the eye for receiving visual signals. Suitable light-sensitive arrays are known in the art, some of which are discussed below.

V. Visual Prosthesis

In certain preferred embodiments of the invention an array comprising a plurality of neural stimulation devices as described above is used as a visual prosthesis, e.g., a retinal prosthesis. A retinal prosthesis may comprise light-sensitive elements such as light-sensitive proton pumps, in which case the prosthesis responds directly to incident light in the environment of the subject. In other embodiments separate light-sensing means are provided. For example, the visual prosthesis may comprise a light-sensitive array for receiving incident light and for generating an electrical signal in response to the incident light. The electrical signal is used to trigger release of the stimulating species, e.g., by triggering collapse of the hydrogel in the storage/release component. Appropriate electronic circuitry is provided to couple the light-sensitive array to the releasing array. An external power source may also be provided. There may also be provided control means for specifying, from outside a body of a patient, a transformation by which electrical signals from the light-sensitive array are transformed into a set of electrical patterns to the releasing array to influence visual function, as described in U.S. Pub. No. 20030158588. The light-sensitive array may comprise, e.g., photosensitive elements of a variety of different types such as photodiodes. Suitable light-sensitive arrays are described, e.g., in U.S. Pat. Nos. 5,895,415 and 5,397,350.

FIG. 1F depicts an exemplary hybrid biochemical/electronic retinal prosthesis 500 that comprises an array 510 (biochemical release array) of neural stimulation devices 10. The electronic components are generally similar to those described in U.S. Pub. No. 20030158588 except that the prosthesis does not include an electrode array for directly stimulating the retina. Instead, stimulation is achieved using the array of neural stimulation devices. The neural stimulation devices are hybrid biochemical/electronic devices as described above and comprise electrodes 92 that interface with the electronic components and receive signals therefrom to trigger release of the stimulating species by application of an electric field. The storage/release component comprises an electric field responsive hydrogel. The prosthesis may comprise a light-sensitive array comprising bacteriorhodopsin thin films or any other light-sensitive element. The light-sensitive elements of the light-sensitive array may be within the release array or may be separate. Thus in certain embodiments the prosthesis comprises a release array that also functions to sense light while in other embodiments a separate light-sensitive array is provided. Elements of the release array and the light-sensitive array may be intermingled in a single array.

The electronic components of the prosthesis comprise three modules that are flexibly connected: a coil and array module 520, a connection module 530, and a control module 540. The coil and array module 520 comprises an RF power coil 540 for receiving power from a power source. The release and light-sensitive array (or separate release array and light-sensitive arrays) are flexibly attached to the power coil by a flexible wire connection bus 550 that is connected to the power coil via the bonding attachment area 560. The flexible wire connection bus has surgical handles 570 and the power coil has surgical handles and/or holes 580 for manipulation of the prosthesis by the surgeon. The design allows for surgical access space within the power coil. The light-sensitive array provides input devices for optical signal to the prosthesis, and the elements of the release array constitute output devices that release a stimulating species to neuron in the retina to stimulate it and thereby convey useful visual information to the patient. In a preferred embodiment, the release array measures 2 mm in length and the power coil has an inner diameter of 6 mm and an outer diameter of 12 mm. The flexible wire connection bus, from the attachment area to the release array measures 10 mm in length. All of these components are ultra-thin, having a height preferably less than 1 mm.

The connection module 530 comprises a flexible bridge for sending the electrical signals to and from a stimulator chip which generally comprises a microprocessor as described above. The connection module is thin and smooth and of a length such that it may be positioned underneath an extraocular muscle for moving the eye without negatively affecting operation of the muscle. In a preferred embodiment, the connection module measures 9 mm in length and 3 mm in width. The control module comprises a stimulator chip or other electronic circuitry 600 for receiving input signals from the light-sensitive array components and controlling the electrical signals delivered to the release array elements for retinal tissue stimulation. The stimulator chip preferably contains rectifier circuitry to rectify the oscillating voltage obtained from the power coil, and the control module preferably further comprises discrete power supply capacitors 610 for smoothing the rectified voltage and delivering it to the stimulator chip. In other embodiments the capacitors are integrated into circuitry 600 rather than being discrete devices.

In a preferred embodiment optical communication from the external world is wireless. The RF secondary coil is the input device for transmission of power to the prosthesis by magnetic coupling from the RF primary coil outside the body. Electrical power from the RF secondary coil electrical signals from the light-sensitive array are sent to the stimulator chip through a set of embedded wires. The stimulator chip receives transformation information from the RF secondary coil through the same set of wires that carry power. The stimulator chip processes the data from the light-sensitive array in accordance with a transformation algorithm and uses this information to apply current pulses to the release array to cause release of the stimulating species to stimulate the retina in a pattern that conveys to the patient useful visual information. In this embodiment, the RF secondary coil also serves as the input device for the communication of a transformation algorithm and parameters of the algorithm by which the optical pattern incident on the light-sensitive array is converted to a pattern of electrical stimulation of the retina that conveys useful information to the patient and influences visual function. It will be appreciated that an external light-sensitive device, e.g., a camera, could be used rather than a light-sensitive array within the eye. Numerous other variations are possible. For example, alternate power sources and arrangements of the same or other electronic componenents are within the scope of the invention.

A retinal prosthesis of the invention can be implanted into the eye of a subject using established methods. In general, an epi-retinal device is placed on or near the inner surface of the retina, that is, the side which is first exposed to incoming light rays and along which the nerve fibers of the ganglion cells pass on their way to the optic nerve. Sub-retinal devices are placed under the retina, between the retina and the underlying retinal pigment epithelium or other deeper tissues. Although devices in either location are capable of effectively stimulating retinal nerve cells, there are advantages and potential disadvantages to each strategy. One very significant advantage of a sub-retinal prosthesis is the opportunity to implant the device by approaching the sub-retinal space from outside of the eye (i.e. ab externo, through the sclera covering the back of the eye), rather than having to perform any (or any significant) surgery within the center of the eye, which is much more likely to result in chronic inflammation, infection or a host of other problems that might compromise the safe implantation or effectiveness of a prosthesis. For this and other reasons, the sub-retinal approach is preferred for the present invention.

While a retinal prosthesis is a preferred embodiment of the present invention, a neural stimulation device according to the present invention may be used to stimulate neural tissue in areas of the visual pathway other than the retina. For example, a device may be implanted elsewhere along the visual pathway, including the optic nerve, primary visual cortex, secondary visual cortices, chiasm, the optic tract, lateral geniculate body, and optic radiations.

VI. Device Fabrication

A device of the invention may be manufactured using a variety of different methods. In general, a device such as neural stimulation device of the invention may be made using methods commonly termed “microfabrication” or “nanofabrication” techniques. Methods useful for implementation of the device may be found in, e.g., U.S. Pat. No. 5,776,748 to Singhvi et al.; U.S. Pat. No. 5,900,160 to Whitesides et al.; U.S. Pat. No. 6,060,121 to Hidber et al.; U.S. Pat. No. 6,180,239 to Whitesides et al.; “Patterning of a Polysiloxane Precursor to Silicate Glasses by Microcontact Printing”, Marzolin, et al., Thin Solid Films 1998, 315, 9-12; “Microfabrication, Microstructures and Microsystems”, Qin, et al.; In Microsystem Technology in Chemistry and Life Sciences, vol. 194, Manz, A. and Becker, H., Eds.; Springer-Verlag, Berlin, 1998, 1-20; “Unconventional Methods for Fabricating and Patterning Nanostructures,” Xia et al., Chem. Rev. 99:1823-1848 (1999).

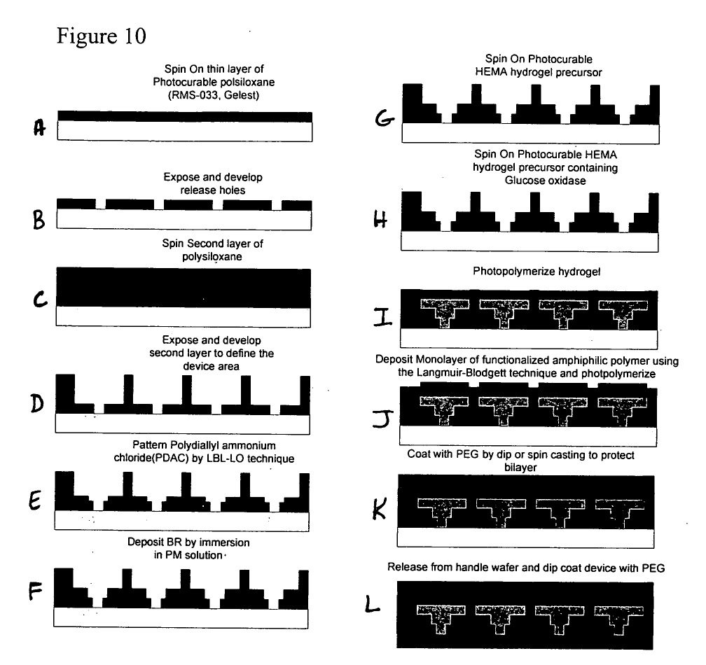

FIG. 10 illustrates an exemplary fabrication procedure for a neural stimulation device for use as a retinal prosthesis. The method can be considered to consist of 12 steps, labeled A-L in the figure. The first step is to spin a thin layer of biocompatible polymer e.g., polysiloxane, onto a substrate and define the release apertures for the storage/release layer photolithographically. This could also be done by chemical vapor deposition of parylene followed by reactive ion etching to define the release apertures. In some cases it may be desirable to make this membrane a proton conducting membrane by doping it with the proton ionophore or to use a polymer such as nafion [74], a perfluorinated polymer which is intrinsically proton conducting due to the superacid side chain. Use of a proton conducting material may help speed up the proton exchange to the light driven proton pump. Alternate proton conduction polymers could also be used.

The polymer capsule that holds the hydrogel that forms the storage/release layer is defined in the second step by spinning on a thick layer of polymer, which is defined either photolithographically or by reactive ion etching. Bacteriorhodopsin, the photon driven proton pump of choice, can be deposited by using the layer-by-layer (LBL) technique [11]. First, polyammonium diallyl dimethylchloride (PDAC), a polycation electrolyte, is deposited and patterned using the layer-by-layer-lift-off (LBL-LO) technique [75, 76]. The polycation layer serves as an anchor and an orientation surface by exploiting the intrinsic excess negative charge such that exists on the cytoplasmic side of BR in solutions with pH greater than 5 [11] and in this orientation light will drive protons into the device. The PDAC coated substrate is then immersed in a solution that contains BR. BR can also be deposited using other techniques such as self assembly [77] or by laser ablation which is more compatible with microfabrication [78].

The photocurable hydrogel layer is spun on in two steps to ensure that the majority of the enzyme concentration, glucose oxidase in this embodiment, is near the uptake component layer. The uptake component layer is then deposited using a Langmuir-Blodgett technique. Briefly, the polymer solution is dissolved in an organic solvent and then spread on the air water interface. The patterned substrate is then slowly dipped to transfer the film onto the substrate. The film formed is then photopolymerized to freeze the structure in place. The device is then dip or spin coated with polyethylene glycol (PEG) to protect the uptake component during handling. The device is then removed from the substrate by gentle peeling in the case of polysiloxane or by removal of a sacrifical layer that could be optionally deposited before the first polymer layer is deposited.

In other embodiments the pH-responsive gel release system is replaced with an electrically collapsible gel. The fabrication is very similar to the one for the all biochemical device described above and is shown in FIG. 11. The method can be considered to consist of 11 steps, labeled A-K in the figure. However, instead of patterning PDAC by lift-off gold or another conductive material is used. The pattern defines both the electrodes and the leads that will be used to connect the biochemical array to the electrical system. Methods for fabrication of electronic components of a hybrid biochemical/electronic device are known in the art. Exemplary methods are described in U.S. Pub. No. 20030158588. Standard silicon manufacturing technology is employed for various of the electrical components.

VII. Therapeutic Applications

In general, the devices and methods of the invention are useful in any of a variety of situations that involve injury or damage to neural cells or neural target cells. Such injury or damage may occur as a result of surgery, trauma, stroke, tumor, neurodegenerative disease, or other diseases or conditions. The devices and methods can also be used in contexts that do not necessarily involve injury or damage to neural cells or neural target cells. Exemplary conditions that can be treated using the devices of the invention include visual impairment, hearing impairment, pain, epilepsy, Parkinson's disease, a neurodegenerative disorder, bowel dysfunction, bladder dysfunction, muscle wasting, stroke, sleep apnea, diaphragmatic dysfunction, myasthenia gravis, multiple scleroris, neuropathy, paresis, and paralysis.

The neural stimulation device can be implanted anywhere within the body, e.g., in the CNS, PNS, in proximity to a muscle, etc. A neural stimulation device in which dopamine is the stimulating species is of particular use for treatment of Parkinson's disease, which involves degeneration of dopamine-secreting neurons in the brain.

The methods and compositions of the invention may be tested using any of a variety of animal models for injury or damage to the nervous system.

### EXAMPLES

### Example 1

Materials and Methods

Single cell recordings were made from the axons of rabbit retinal ganglion cells in vitro. Data was recorded and analyzed using Spike2 (Cambridge Electronic Design). The ganglion cells were stimulated over the optical receptive field with a multibarrel micropipette (7-barrel, FHC, Inc.) which contained various concentrations of KCl (0-30 mM in an osmotically balanced NaCl solution (˜300 mOsm). The micropipette solutions were ejected by using a mulitchannel pressure ejector (PM8000, MDI Systems). All solutions contained Azure B (0.5 mg/ml) to enable visualization of the solution being ejected. Pulse durations of 20-100 msecs were used.

Experimental Results

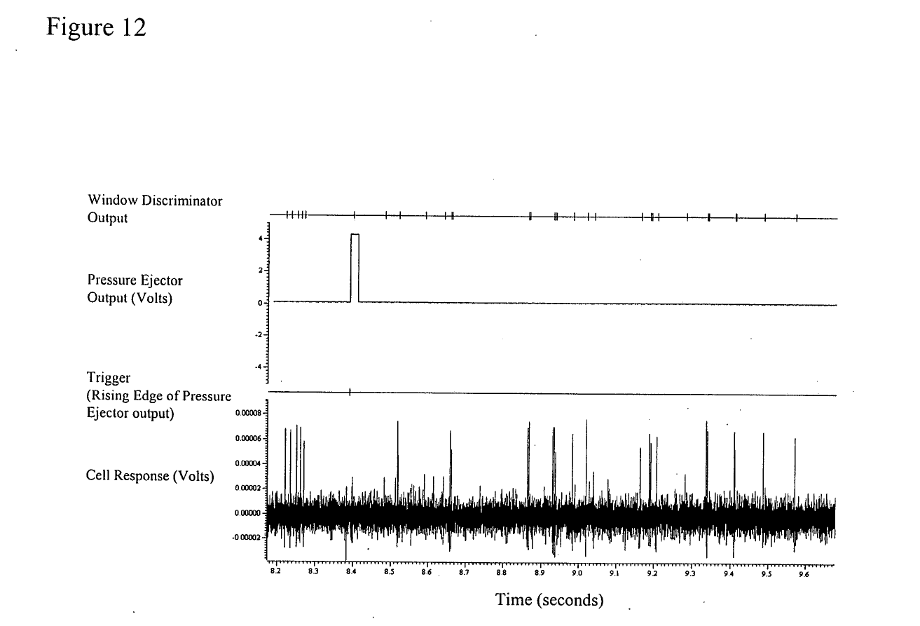

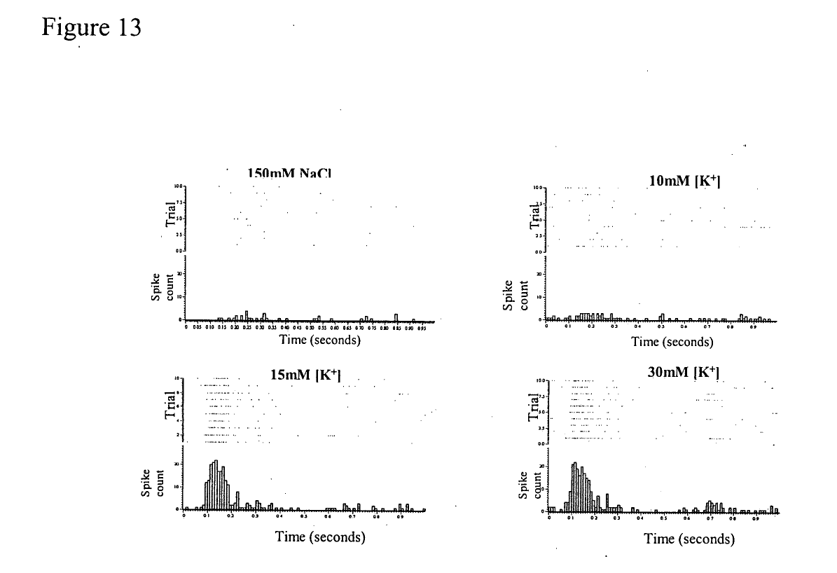

FIG. 12 shows a typical record from a single cell. Response is shown for a cell stimulated with a 20 msec pulse of 10 mM K⁺, pressure of 40 p.s.i. and a volume of approximately 100 pL. FIG. 13 shows peri-stimulus histograms (PSTH) for a representative cell stimulated by increasing [K+] concentrations. A modest increase (10 mM) in K⁺ concentration increases the spike count from 35 to 80 spikes. Further increases in concentration to 15 mM and 30 mM increases the spike rate to 240 and 272 spikes, respectively. The response shows the saturating characteristics indicated in the dose-response curve. This may be due to the fact that the retina is over stimulated by higher concentrations and exhibits a refractory behavior. The histograms are summed responses from 10 trials as indicated by the raster plot.

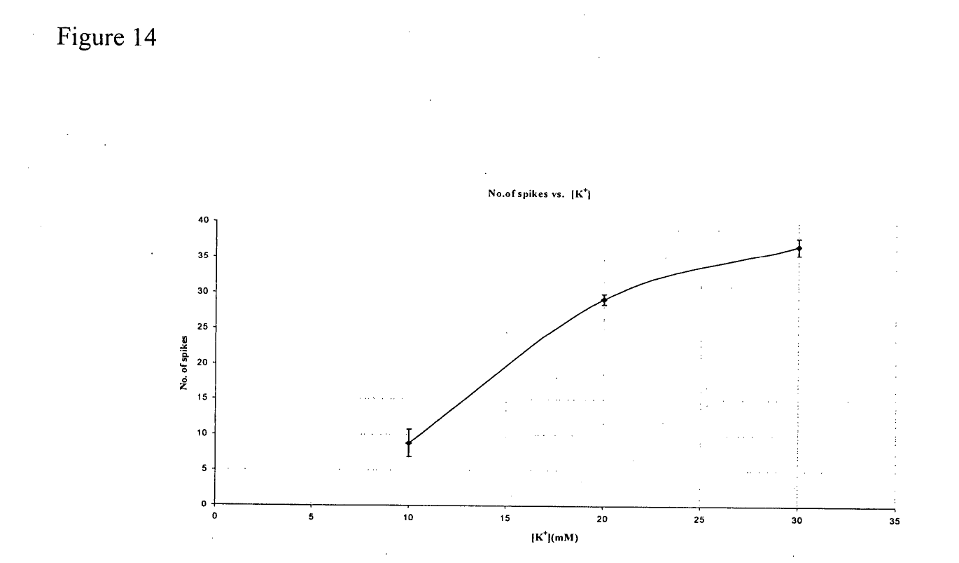

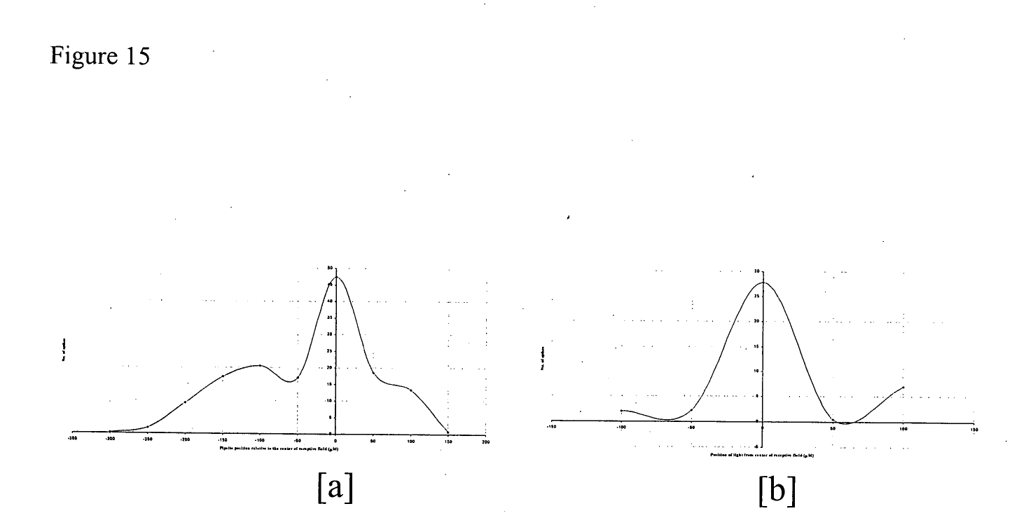

FIG. 14 shows dose-response curves for various concentrations. Data was collected from 5 cells and the total spike count was obtained from the PSTH. The spontaneous activity (control record) was subtracted, divided by the number of repetitions and the median is plotted. The error bars shown are the standard deviations normalized by the median.

FIG. 14 shows receptive field of a retinal ganglion cell following: a) Stimulation by elevated extracellular potassium (30 mM [K+]); b) Stimulation by a spot of light. The receptive fields are similar in size and shape for both light and potassium evoked responses. The large secondary lobe in the potassium response may be due to the fluid flow pattern that may have stimulated adjacent cells. Overall receptive field size is 400 mm (100 mm for primary lobe) for a potassium evoked response and 100 mm for a light evoked response.

The foregoing description is to be understood as being representative only and is not intended to be limiting. Alternative systems and techniques for making and using the compositions and devices of the invention and for practicing the inventive methods will be apparent to one of skill in the art and are intended to be included within the accompanying claims.

### REFERENCES

1. “Potassium-evoked directionally selective resposes from rabbit retinal ganglion cells”, Ralph J. Jensen, Visual Neuroscience, 1996, 13, 705-719.
2, “Membrane Potential in pinched-off presynaptic nerve terminals monitored with fluorescent probe: evidence that synaptosomes have potassium diffusion potentials”, M. P. Blaustein and J. M. Goldring, J. Physiology (Lond.) 247, 589-615, 1975.
3. “External Ca²⁺-Independent Release of Neurotransmitters” Vera-Adam-Vizi, Journal of Neurochemistry, 395-405, 1992.
4. “Molecular Basis for the Action of Macrocyclic Carriers on Passive Ionic Translocation Across Lipid Bilayer Membranes”, G. Eisenmann, G. Szabo, S. G. A McLaughlin and S. M. Ciani, Bioenergetics, 4, 93-148, 1973
5. “Chapter 6: Carrier Mediated Transport”, pp. 333-447, Cellular Biophysics-Transport, Vol. 1. Thomas F. Weiss, 1996, MIT Press
6. “Polymerized ABA Trblock Copolymer Vesicles”, C. Nardin, T. Hirt, J. Leukel and W. Meier, Langmuir 2000, 16, pp. 1035-1041.
7. “Giant Free-Standing ABA Triblock Copolymer Membranes”, C. Nardin, M. Winterhalter, W. Meier, Langmuir 2000, 16, pp. 7708-7712.
8. “Reconstitution of Channel Proteins in (Polymerized) ABA Triblock Copolymer Membranes”, W. Meier, C. Nardin and M. Winterhalter, Angew. Chem. Int. Ed. 2000, 39, No. 24, 4599-4601.
9. “Synthesis and Characterization of Polysiloxane-Bound Receptor Molecules for Ion-Selective Supported Membranes”, H-A. Klok, P. Eibeck, M. Moller and D. N. Reinhoudt, Macromolecules, 1997, 30, 795-802.
10. “Development of Durable K+-Selective Chemically Modified Field Effect Transistors with Functionalized Polysiloxane Membranes”, D. N. Reinhoudt, J. F. Engbersen, Z. Brsoska, H. H. Van den Vlekkert, G. Q. N. Honig, H. A. J. Holterman, U. H. Verkerk., Analytical Chemistry, Vol. 66, No. 21, Nov. 1, 1994.
11. “Bacteriorhodopsin Thin Film Assemblies-Immobiilzation, Properties and Applications”—Jin-An He, Lynee Samuelson, Lian Ji, Jayant Kumar and Sukhant K. Tripathy, Advanced Materials, 1999, 11, No. 6, 1-11.
12. “Fast pH- and Ionic Strength-Responsive Hydrogels in Microchannels”, Bin Zhao and Jeffrey S. Moore, Langmuir 2001, 17, 4758-4763.
13. “Rapid Deswelling Response of Poly(N-isopropylacrylamide) Hydrogels by the Formation of Water Release Channels Using Poly(ethylene oxide) Graft Chains”, Yuzo Kaneko, Satoki Nakamura, Kiyotaka Sakai, Takao Aoyagi, Akihiko Kikuchi, Yasuhisa Sakurai, and Teruo Okano, Macromolecules 1998, 31, 6099-6105.
14. “Nonvesicular release of neurotransmitter”, D. Atwell, B. Barbour and M. Szatkowski, Neuron, Vol. 11, 401-407, September, 1993.
15. “Non-vesicular release of glutamate from glial cells by reversed electrogenic glutamate uptake” M. Szatkowski, B. Barbour and D. Atwell.
16. “Muller Glial Cells in the Anuran Retina”, A. Bringmann, S, Skatchkov, T. Pannicke, B. Biedermann, H. Wolburg, R. K. Orkand and A Reichenbach, Microscopy Research and Technique, 50:384-393, 2000.
17. “Immobilization of Crown Ether Carboxylic Acids on Silica Gel and Their Use in Column Concentration of Alkali Metal Cations from Dilute Aqueous Solutions”, Matthew G. Hankins, Takashi Hayashita, Stanislaw P. Kasprzyk, and Richard A. Bartsch, Anal. Chem. 1996, 68, 2811-2817
18. “Influence of pH gradients on the transbilayer transport of drugs, lipids, peptides and metal ions into large unilamellar vesicles”, Biochimica et Biophysica Acta 1331, 1997, 187-221.
19. “Structure, Conformation, and Mechanism in Membrane transport of Alkali Metal Ions by lonophoric Antibiotics” Frank G. Riddell, Chirality, 14, 121-125, 2002.
20. “The Nigericin-Mediated Transport of Sodium and Potassium Ions through Phospholipid Bilayers Studied by 23Na and 39K NMR Spectroscopy” Frank G. Riddell, t S. Ammugam, Peter J. Brophy,* Brian G. Cox, Mary C. H. Payneand, Timothy E. Southonts, J. Am. Chem. Soc. 1988, 110, 734-738.
21. “Antibiotic Mediated Transport of Alkali Ions across Lipid Barriers” B. C. Pressman, E. J. Harris, W. S. Jagger and J. H. Johnson., Vol. 58. Issue 5, Proc. Natl. Acad. Sci. 1949-1956, 1967.
22. “Theoretical and Experimental Basis for a Specific Countertransport System in Membranes” E. L. Cussler, D. Fennell Evans, Sister M. A. Matesich, Science, Vol 172, 3981, 1971, 377-179.
23. “Enhancement of Uphill Transport by a Double Carrier Membrane System” M. Sugawara, M. Omoto, H. Yoshida and Y. Umezawa, Anal. Chem. 1988, 60, 2301-2303
24. “Uphill transport Membrane Systems”, M. Uto, M. Sugawara, H. Yoshida and Y. Umezawa Anal; Chem, 1986, 58, 1798-1803.
25. “Proton Ionizable Crown Compounds: 5. Macrocycle-mediated proton coupled transport of alkali metal cations in H₂O—CH₂Cl₂—H₂O liquid membrane systems” R. M. Izatt, G. C. Lindh, G. A. Clark, Y. Nakatsuji, J. S. Bradshaw, J. D. Lamb and J. J. Christensen, Journal of Membrane Science, 31, 1987, 1-13.
26. “Selective Column Concentration of Alkali-Metal Cations with a Crown Ether Carboxylic Acid Resin” Takashi Hayashita, Joung Hae Lee, Shiping Chen, and Richard A. Bartsch, Anal. Chem. 1991, 63, 1844-1847.
27. “Solvent Extraction of Alkali Metal Cations from Aqueous Solutions by Highly Lipophilic Crown Ether Carboxylic Acids” Jerzy Strzelblcki and Richard A. Bartsch, Anal. Chem. 1981, 53, 2251-2253.
28. “Chemiosmotic coupling in oxidative and photosynthetic phosphorylation.” Mitchell P., Biol. Rev. Camb. Phil. Soc. 41, 445-502 (1966).
29. “Membrane Transport System. I. Synthesis and characterization of carriers for a proton driven alakali metal cation pump” L. A. Fredrick, T. F. Fyles, N. P. Guruprasad and D. M. Whitefield, Can. J. Chem. 59, 1724-1733, 1981
30. “Membrane Transport Systems. II. Transport of alkali metal ions against their concentration gradients”, T. M. Fyles, V. A. Malik-Diemer, and D. M. Whitfield, Can. J. Chem. 59, 1734-1744, 1981
31. “Membrane Transport Systems. III. A mechanistic study of cation-proton coupled countertransport”, T. M. Fyles, V. A. Malik-Diemer, C. A. McGavin and D. M. Whitfield, Can. J. Chem. 60, 2259-2267, 1982
32. “Membrane Transport Systems. VI. Complexation of cations by lipophilic crown ether carboxylic acids”, T. M. Fyles and D. M. Whitfield, Can. J. Chem. 62, 507-514, 1984
33. “Membrane Transport Systems. V. Coupled countertransport of alkaline earth cations and protons”, L. M. Dulyea, T. M. Fyles and D. M. Whitfield, Can. J. Chem. 62, 498-506, 1984
34. “Complexing and chelating agents immobilized on silica gel and related materials and their application for sorption of inorganic species”, J. F. Biemat, P. Konieczka, B. J. Tarbet, J. S. Bradshaw and R. M. Izatt, Sep. Pur. Meth., 23(2), 77-348. 1994.
35. “Novel Lipophilic Crown carboxylic acids”, B. Czech, B. Son and R. A. Bartsch, Tetrahedron Letters, Vol. 24, No. 29, pp. 2923-2926, 1983.
36. “Ion Transport across Membranes Prepared by Gel Crystallization”, H-A. Klok, P. Eibeck, H. Gankema, R. P. Nieuwhof, M. Moller, D. N. Reinhoudt, Journal of Polymer Science: Part B: Polymer Physics, Vol. 36, 383-394 (1998).
37. “The Purple Membrane of Salt-loving Bacteria”, Walther Stoeckenius, Scientific American, 1976, 234(6), 38-46.
38. “The Photosynthetic reaction center from the Purple bacteria Rhodopseudomonas viridis”, J. Deisenhofer, H. Michel, Sciene, Vol. 245, 4925, Sep. 29, 1989 1463-1473
39. “Amperometric glucose biosensor based on redox polymer-mediated electron transfer”, P. D. Hale, L. I. Boguslavsky, T. Inagaki, H. I. Karan, H. S. Lee, T. A. Skotheim and Y. Okamoto, Anal. Chem, 1991, 63, 677-682.
40. “pH-based enzyme potentiometric sensor. Part 2. Glucose-sensitive field effect transistor”, S. D. Caras, D. Petelenz and J. Janata, Anal. Chem., 1985, 57, 1920-1923.
41. “Catalase Effects on Glucose-Sensitive Hydrogels”, Dal-Young Jung, Jules J. Magda, and In Suk Han, Macromolecules 2000, 33, 3332-3336.
42. “Bio-smart hydrogels: co-joined molecular recognition and signal transduction in biosensor fabrication and drug delivery”, Sean Brahim, Dyer Narinesingh, Anthony Guiseppi-Elie, Biosensors and Bioelectronics 17 (2002) 973-981
43. “Selective monosaccharide transport through lipid bilayers using boronic acid carriers”, p. R. Westmark, S. J. Gardiner and B. D. Smith, J. Am. Chem. Soc., 1996, 118, 11093-11100.
44. “Enzyme Immobilization by condensation Co-polymerization into cross-linked polyacrylamide gels”, A. Pollack, H. Blumenfeld, M. Wax, R. L. Baughn and G. M. Whitesides, J. Am. Chem. Soc. 1980, 102, 6324-6336.
45. “Synthesis and hydroation properties of PH-Sensitive p-(HEMA)-based hydrogels containing 3-(trimethylsilyl)propyl methacrylate”, Sean Brahim, Dyer Narinesingh and Anthony Guiseppi-Elie, Biomacromeolecules, 2003, 4, 497-503.
46. “Equilibrium Swelling and Kinetics of pH-Responsive Hydrogels: Models, Experiments, and Simulations”, Sudipto K. De, N. R. Aluru, B. Johnson, W. C. Crone, David J. Beebe, and J. Moore, J. MEMS, vol. 11, no. 5, October 2002, 544-555
47. “A universal on-wafer fabrication technique for diffusion limiting membranes for use in microelectrochemical amperometric sensors” A. van den Berg, A. grisel, M. Koudelka and B. H. van der schoot, Sensors and Actuators B, 5(1991) 71-74.
48. “Fabrication and Characterization of Hydrogel-Based Microvalves” R. H. Liu, Q. Yu and D. J. Beebe, J. MEMS, Vol. 11, No 1. February 2002, 45-53
49. “Proton Transfer Pathways in Bacteriorhodopsin at 2.3 Angstrom Resolution.” H Luecke, H-T Richter & J K Lanyi (1998) Science 280, 1934-1937
50. “The Biophysics of Visual Photoreception”—Aaron Lewis and Lucian V. Del Priore, Physics today, January 1988, 3846
51. “The lipid bilayer concept and its experimental realization: from soap bubbles, kitchen sink, to bilayer lipid membranes”, H. T. Tien, A. L. Ottova, J. Mem. Sci., 189 (2001) 83-117.
52. “Supported planar lipid bilayers (s-BLMAs) as electrochemical biosensors: H. T. Tien and A. L. Ottava, Electrochica Acta. Vol 43, No. 23, pp. 3587-3610, 1998.
53. Yilgor et al “Reactive Difunctional Siloxane Oligomers: Synthesis and Characterization”, Reactive Oligomers, ACS, 1985, 162-174.
54. “Two Reducible Protecting Groups for Boronic Acids”, Christophe Malan, Christophe Morin and Gatan Preckher, Tetrahedron Letters, Vol. 37, No. 37, pp. 6705-6708, 1996.
55. Lindskog, S., Henderson, L., Kannan, K., Liljas, A., Nyman, P., and Strandberg, B.: Carbonic Anhydrase, The Enzymes 5, 587, 1971.
56. “Lasalocid A facilitated dopamine uphill transport and concentration into liposomes driven by pH gradient generated by bacteriorhodopsin under illumination”, Zhe Quan, Masao Sugawara, Yoshio Umezawa, Biosensors & Bioelectronics 13 (1998) 1157-1164
57. “Selective Dopamine Transport Using a Crown Boronic Acid”, Marie-France Paugam, Linbee S. Valencia, Bill Boggess, and Bradley D. Smith, J. Am. Chem. SOC. 1994, 116, 11203-11204.
58. “Facilitated Catecholamine Transport through Bulk and Polymer-Supported Liquid Membranes”, Marie-France Paugam, Jeffrey T. Bien, Bradley D. Smith, Lysander A. J. Chrisstoffels, Feike de Jong, and David N. Reinhoudt, J. Am. Chem. Soc. 1996, 118, 9820-9825.
59. “Synthesis of an Ammonium Ionophore and Its Application in a Planar Ion-Selective Electrode”, John S. Benco, Hubert A. Nienaber, and W. Grant McGimpsey, Anal. Chem. 2003, 75, 152-156.
60. Gutsche, C. D. Calixarenes Revisited, Monographs in Supramolecular Chemistry; Stoddard, J. F., Ed.; The Royal Society of Chemistry, Cambridge, 1998.
61. “Novel Triblock Siloxane Copolymers: Synthesis, Characterization and Their Use as Surface Modifying Additives”, I. Yilgor, W. P. Steckle, E. Yilgor, R. G. Freelin and J. Riffle, Journal of Polymer Science: Part A: Polymer Chemistry, Vol. 27, 3673-3690, 1989.
62. “Asymmetric Amphiphilic Triblock Copolymers: Synthesis, Characterization and Self-assembly”, Roxana Stonescu, Ph. D. Thesis, Basel University, 2004.
63. Pederson, C. J. J. Am. Chem. Soc. 89, 2498, 7017 (1967).
64. Gokel G W, Leevy W M, Weber M E., Crown ethers: sensors for ions and molecular scaffolds for materials and biological models. Chem Rev. 104 (5):2723-50 (2004).
65. Cram, D. J.; Cram, Accts. Chem. Res. 11, 8 (1978).
66. Krakowiak, K E, et al, Chem. Rev. 89, p. 929, 1989.
67. Bradshaw, J S, et al., Aza-Crown Compounds; Wiley: New York, 1993, Vol. 51, 1993.
68. “Electrical/pH Responsive Properties of Poly(2-acrylamido-2-methylpropane sulfonic acid)/Hyaluronic Acid Hydrogels”, Seon Jeong Kim, Chang Kee Lee, Sun I. Kim, Inc. J Appl Polym Sci 92:1731-1736, 2004
69. “Properties of smart hydrogels composed of polyacrylic acid/poly(vinyl sulfonic acid) responsive to external stimuli”, Seon Jeong Kim, Sang Jun Park and Sun I Kim, Smart Mater. Struct. 13 (2004) 317-322
70. “Collapse of Gels in an Electric Field”—Toyichi Tanaka, Izumi Nishio, Shao-Tang Sun, Shizue Ueno-Nishio, Science, Vol. 218, Oct. 29, 1982, 467-469.
71. “Multipoint Molecular Recognition of Amino Acids and Biogenic Amines by Ureidocalix[5]arene Receptors”, Ballistreri, F. P., et al., Organic Letters, Vol 5, No. 7, 1071-1074, 2003.
72. “Transport of Amino Acids Through Organic Liquid Membranes”, Behr, J. and Lehn, J-M., J. Am. Chem. Soc., 95:18, 1973.
73. “Crown-Ether-Functionalized Mn(III) Salicylaldimine Complexes as Ditopic Carriers. Efficient Transport of Tryptophan and Serotonin across a CHCl₃ Barrier”, Rosa, D. T., et al., Inorg. Chem., 37, 5042-5043, 1998.
74. “Protein-driven energy transduction across polymeric biomembranes”, Dean Ho1, Benjamin Chu1, Hyeseung Lee1 and Carlo D Montemagno, Nanotechnology 15 (2004) 1084-1094
75. “Lithographic Approach to Pattern Self-Assembled Nanoparticle Multilayers”, Feng Hua, Tianhong Cui, and Yuri Lvov, Langmuir 2002, 18, 6712-6715.
76. “Fabrication of 3-D Gelatin-Patterned Glass Substrates With Layer-by-Layer and Lift-Off (LbL-LO) Technology”, Mengyan Li, Kishore K. Kondabatni, Tianhong Cui, and Michael J. McShane, IEEE Trans. Nanotechnology, Vol. 3, No. 1, March 2004.
77. “Photoelectric conversion of bacteriorhodopsin films fabricated by self-assembly technique”, Hyun-Goo Choia, Junhong Mina, Keon-Kook Hana, Jeong-Woo Choia, Won Hong Leea, Synthetic Metals 117 (2001) 141-143.
78. “Microfabricating bacteriorhodopsin films for imaging and computing”, Dan Haronian and Aaron Lewis, Appl. Phys. Lett. 61 (18), 2 Nov. 1992.
79. Highly Fructose Selective Transport Promoted by Boronic Acids Based on a Pentaerythritol Core, Draffin, S. P.; Duggan, P. J.; Duggan, S. A. M., Org. Lett.; 2001; 3(6); 917-920.

We claim:

1. A neural stimulation device comprising:
(i) an uptake component comprising means for selectively transporting a first species into the device, wherein the first species is a stimulating species;
(ii) a release component comprising means for releasing the stimulating species; and
(iii) means for producing a concentration gradient of a second species, wherein the concentration gradient of the second species provides energy to transport the stimulating species into the device.

2. The device of claim 1, wherein the stimulating species is an ion or neurotransmitter.

3. The device of claim 1, wherein the stimulating species is a potassium ion.

4. The device of claim 1, wherein the stimulating species is a catecholamine.

5. The device of claim 1, wherein the second species is a proton.

6. The device of claim 1, wherein the means for selectively transporting a first species into the device comprises a crown ether.

7. The device of claim 1, wherein the means for selectively transporting a first species into the device comprises a carboxylic acid or a boronic acid.

8. The device of claim 1, wherein the uptake portion comprises a membrane having two hydrophilic layers separated by a hydrophobic layer.

9. The device of claim 8, wherein the membrane comprises a synthetic polymer.

10. The device of claim 8, wherein the membrane comprises an ABA triblock polymer.

11. The device of claim 8, wherein the means for selectively transporting a first species into the device comprises a carrier molecule that is covalently attached to the membrane.

12. The device of claim 1, wherein the means for selectively transporting a first species into the device comprises a carrier molecule that transports the stimulating species and the second species across the uptake component in opposite directions.

13. The device of claim 1, wherein the means for producing a concentration gradient of a second species comprises a proton pump that transports protons into the device or an enzyme that catalyzes a chemical reaction that produces protons in the device.

14. The device of claim 13, wherein the means for producing a concentration gradient of a second species comprises a glucose carrier.

15. The device of claim 1, wherein the means for producing a concentration gradient of a second species comprises a proton pump or an enzyme that catalyzes a chemical reaction that generates protons inside the device.

16. The device of claim 1, wherein the means for releasing the stimulating species comprises a stimulus-responsive hydrogel.

17. The device of claim 16, wherein the hydrogel undergoes a change in volume in response to a change in pH or in response to a voltage or current.

18. The device of claim 1, wherein the device comprises a storage component that stores the stimulating species, wherein the storage component is either distinct from the release component or wherein a single component both stores and releases the stimulating species.

19. The device of claim 1, further comprising:
means for receiving an activating input; and
means for coupling receipt of the activating input to release of the stimulating species.

20. The device of claim 19, wherein the means for receiving an activating input and means for coupling receipt of the activating input to release of the stimulating species comprises a light-sensitive proton pump that transports protons into the device in response to light.

21. The device of claim 19, wherein the means for receiving an activating input, the means for coupling receipt of the activating input to release of the stimulating species, or both, comprises electronic circuitry.

22. The device of claim 21, wherein the electronic circuitry comprises a light-sensitive electronic component.

23. The device of claim 22, wherein the light-sensitive electronic component is a photodetector.

24. The device of claim 19, further comprising means for generating an activating stimulus.

25. The device of claim 24, wherein the means for generating an activating stimulus comprises (i) a computer, (ii) a transducer that transduces sound, pressure, heat, or motion, into an electrical signal; or (iii) both.

26. The device of claim 22, further comprising:
an external power source; and
means to receive power from the external power source.

27. The device of claim 26, wherein the external power source provides power to trigger release of the stimulating species.

28. A method of treating a subject in need of neural stimulation comprising implanting the device of claim 1 into the subject.

29. The method of claim 28, wherein the subject is in need of treatment for a condition selected from the group consisting of: visual impairment, hearing impairment, pain, epilepsy, Parkinson's disease, a neurodegenerative disorder, bowel dysfunction, bladder dysfunction, muscle wasting, stroke, sleep apnea, diaphragmatic dysfunction, myasthenia gravis, multiple scleroris, neuropathy, paresis, and paralysis.

30. A neural prosthesis comprising a release array comprising a plurality of devices as set forth in claim 1, wherein the devices release a stimulating species.

31. The neural prosthesis of claim 30, further comprising a biocompatible substrate layer that provides mechanical support for the devices that comprise the release array.

32. The neural prosthesis of claim 30, wherein each device comprises:
means for receiving an activating input; and
means for coupling receipt of the activating input to release of the stimulating species.

33. A retinal prosthesis comprising a stimulating array comprising a plurality of devices as set forth in claim 1.

34. The retinal prosthesis of claim 33, further comprising a biocompatible substrate layer that provides mechanical support for the devices that comprise the release array.

35. The retinal prosthesis of claim 34, wherein the biocompatible substrate layer is sufficiently flexible to conform to the curvature of the eye.

36. The retinal prosthesis of claim 33, wherein each device comprises:
means for receiving incident light; and
means for coupling receipt of the incident light to release of the stimulating species.

37. The retinal prosthesis of claim 33, further comprising:
a light sensitive array electrically connected to the stimulating array for receiving incident light and generating a signal in response thereto.

38. The retinal prosthesis of claim 33, further comprising:
an external power source; and
means to receive power from the external power source.

39. A method of treating a subject in need of treatment for visual impairment comprising implanting the retinal prosthesis of claim 33 into the subject.

40. A method of accumulating a stimulating species inside a neural stimulation device comprising transporting the stimulating species into the device using energy obtained from transport of a second species from inside the device to outside the device down its concentration gradient.

41. The method of claim 40, wherein the stimulating species is an ion or a neurotransmitter.

42. The method of claim 40, wherein the second species is a proton.

43. The method of claim 40, wherein a carrier molecule transports the stimulating species into the device and transports the second species out of the device.
